SEQUENCE LISTING (6 sequences)

SEQ ID NO: 1 - DNA, 90 bp, Artificial
  Oligonucleotide primer for amplification of mRNA to cDNA
  MISC_FEATURE (89)..(89): v can be a, g, or c
  MISC_FEATURE (90)..(90): n can be a, g, c, or t
aagcagtggtaacaacgcagagtactttttttttttttttttttttttttttttttttttttttttttttttttttttttttttttttvn

SEQ ID NO: 2 - DNA, 90 bp, Artificial
  Oligonucleotide primer for amplification of mRNA to cDNA
  MISC_FEATURE (89)..(89): v can be a, g, or c
  MISC_FEATURE (90)..(90): n can be a, g, c, or t
cgataaaagctccggggataacagatttttttttttttttttttttttttttttttttttttttttttttttttttttttttttttttvn

SEQ ID NO: 3 - DNA, 30 bp, Artificial
  Oligonucleotide primer for amplification of mRNA to cDNA
aagcagtggtaacaacgcagagtacgcggg

SEQ ID NO: 4 - DNA, 46 bp, Artificial
  Oligonucleotide primer for amplification of mRNA to cDNA and incorporating T7 polymerase binding site into cDNA
taatacgactcactatagggaggaagcagtggtaacaacgcagagt

SEQ ID NO: 5 - DNA, 100 bp, Artificial
  Single-stranded sense oligonucleotide probe for capture of human ubiquitin mRNA.
gagaacgtcaaggcaaagatccaggacaaggaaggcattcctcctgaccagcagaggttgatctttgccggaaagcagctggaagatgggcggaccctgt

SEQ ID NO: 6 - DNA, 100 bp, Artificial
  Single-stranded antisense oligonucleotide probe for capture of human ubiquitin mRNA.
acagggtccgcccatcttccagctgctttccggcaaagatcaacctctgctggtcaggaggaatgccttccttgtcctggatctttgccttgacgttctc

(19) United States
(12) Patent Application Publication
(10) Pub. No.: US 2007/0248578 A1
(43) Pub. Date: Oct. 25, 2007

(54) Mrna Transfected Antigen Presenting Cells

(75) Inventor: Irina Tcherepanova, Rougemont, NC (US)

Correspondence Address: MERIX BIOSCIENCE, INC., 4233 TECHNOLOGY DRIVE, DURHAM, NC, 27704, US

(73) Assignee: Argos Therapeutics, Inc., Durham, NC (US)

(21) Appl. No.: 10/580,597

(22) PCT Filed: Nov. 24, 2004
(86) PCT No.: PCT/US04/39535

§ 371 Date: Feb. 27, 2007

Related U.S. Application Data
(60) Provisional application No. 60/525,076, filed on Nov. 25, 2003.

(51) Int. Cl. C07H 21/04; A61K 48/00

(52) U.S. Cl. 424/93.21; 536/23.5

(57) ABSTRACT

A method of amplifying RNA to obtain RNA molecules that are predominantly in the sense orientation and essentially devoid of RNA molecules that are in the anti-sense orientation. A method of transfecting antigen presenting cells with a composition comprising sense RNA encoding immunogenic antigens and essentially devoid of antisense RNA and dsRNA is also provided as well as dendritic cells prepared according to the method.

### RELATED APPLICATIONS

This application claims the benefit of U.S. Provisional Patent Application Ser. No. 60/525,076, filed Nov. 25, 2003, the disclosure of which is incorporated herein by reference in its entirety.

### TECHNICAL FIELD

The presently disclosed subject matter relates to a method of amplifying RNA to obtain RNA molecules that are predominantly in the sense orientation or in the anti-sense orientation, depending on the desired function. A method of transfecting antigen presenting cells with sense RNA encoding tumor antigens or microbial antigens is also provided, as well as antigen presenting cells prepared according to the presently disclosed method.

### BACKGROUND ART

T lymphocytes play an important role in the immune response to infectious agents and tumor cells, as well as in organ transplant rejection and autoimmunity. T cells recognize antigen only when the antigen is presented to them in the form of small fragments bound to MHC molecules on the surface of an antigen presenting cell. In order for the T cells to respond, two signals must be provided by antigen-presenting cells (APCs) to resting T lymphocytes. The first signal, which confers specificity to the immune response, is transduced via the T cell receptor (TCR) following recognition of foreign antigenic peptide presented in the context of the major histocompatibility complex (MHC). The second signal induces T cells to proliferate and become functional. Thus antigen presentation is an important step in the induction of an immune response.

Various approaches have been used to deliver antigen to APCs in an attempt to induce an immune response to that particular antigen. These include the use of viral vectors, insertion of antigen in liposomes, and pulsing cells directly with purified antigens or recombinant proteins.

More recently an antigen delivery system based on the transfection of APCs with RNA has been described. Methods for the treatment or prevention of cancers and pathogen infection using antigen-presenting cells loaded with RNA are disclosed in U.S. Pat. Nos. 6,306,388 and 5,853,719 and related patents and applications, incorporated herein by reference. Briefly, total tumor RNA or selected RNA encoding tumor antigens are amplified and transfected into dendritic cells. Antigen presenting cells transfected with pathogen-derived RNA can also be used for the treatment of infectious diseases.

Antigen-modified dendritic cell-based vaccines hold the great promise for effective anti-tumor immunization, and RNA is widely accepted as a vehicle for antigen delivery to dendritic cells (DCs) (Mitchell and Nair, 2000; Nair and Boczkowski, 2002; Ponsaerts et al., 2003; and Ponsaerts et al., 2002). Tumor RNA encoding for the antigens overcomes limitations inherent to other methods of antigen delivery as it offers the broadest possible tumor antigen repertoire, requiring neither specific HLA haplotypes nor identification and characterization of defined tumor antigens present in the total RNA population.

It is now firmly established that RNA-transfected DCs can stimulate primary cytolytic T cell (CTL) responses (Boczkowski et al., 1996). Numerous studies have confirmed the feasibility of this approach in various models. One study with total renal cell carcinoma RNA-transfected DCs demonstrated effective stimulation of T cell reactivity against primary and metastatic tumors (Heiser et al., 2001 a). Other in vitro studies demonstrate that DCs loaded with autologous tumor RNA can induce specific CTL against bladder cancer (Schmitt et al., 2001), multiple myeloma (Milazzo et al., 2003), breast carcinoma (Müller et al., 2003) and colorectal cancer (Nencioni et al., 2003). Clinical trials that tested vaccination strategies using DCs loaded with autologous total tumor RNA have been carried out for colorectal cancer (Rains et al., 2001), renal cell carcinoma (Su et al., 2003) and malignant glioma (Kobayashi et al., 2003). Only a small portion, 2-3% of total RNA, corresponds to mRNA or antigen encoding species. Enrichment of RNA for mRNA increases the proportion of coding RNA species in the same mass of RNA delivered to the DC. One way of enriching for the coding RNA species is isolation of polyA+RNAs using methods of physical separation. Methods of generating clinical grade DC-based vaccine transfected with cell prostate cell line mRNA have been described (Mu et al., 2004) and clinical trials using the developed approach were initiated (Mu et al., 2003). Another way of enriching the coding RNA population is amplification of RNA biasing amplification of only polyadenylated species. Studies were performed to demonstrate that RNA can be amplified to large amounts from small tumor specimens to enrich for coding species and that occurred without loss of biological function (Boczkowski et al., 2000). In a study aimed at determining whether RNA amplified from a total tumor RNA still retains its biological activity, murine DCs transfected with amplified RNA from a melanoma cell line resulted in antitumor CTL responses. The amplification protocol for RNA described in this study was also applied to prostate tumor cells from miscrodissected laser sections where tumor-specific CTL induction was reported using autologous patient material (Heiser et al., 2001 b). Therefore, use of tumor RNA as a source of antigen offers the further advantage of requiring only small amounts of input tumor tissue from which it could be extracted and amplified.

The conversion of mRNA (polyA+RNA) to complementary DNA and the subsequent amplification of the sequences is a basic technology well known to those skilled in the field of molecular biology. Descriptions of the various permutations of the basic techniques can be found in U.S. Pat. Nos. 5,962,271, 5,962,272 and 6,593,086, the contents of which are hereby incorporated by reference. For example, published protocols for RNA amplification utilize template switching technology (Chenchik et al., 1998). Schematic representation of the technology is demonstrated in FIG. 1A. This technology utilizes a unique property of an RNAseH-deficient mutant of MMLV reverse transcriptase to incorporate additional residues, primarily cytosine at the 5′ end of the synthesized first strand cDNA. If an oligonucleotide containing several consecutive G residues at its 3′ end is present in the first strand synthesis reaction, Watson-Crick base pairing will occur between the G and C nucleotides. Once the reverse transcriptase reaches the 5′ end of the transcript, it switches templates and continues transcription from the defined sequence of the annealed oligonucleotide. The 3′end of the cDNA is defined by annealing poly-dT oligonucleotides that contain other defined sequences. The defined 3′ and 5′ ends of the first strand cDNA will allow for unlimited PCR-based amplification. To allow for subsequent transcription and translation of the antigen sequences in the amplified product, T7 RNA polymerase promoter sequences are introduced by addition of T7 promoter sequences to the oligonucleotide containing G (capswtich) used in the PCR reaction. The template switching protocol originally described by Chenchik et al. was designed for ease of operation, and subsequent subcloning into plasmid vectors.

In this protocol, both the capswitch and poly-dT oligonucleotides contain redundant sequences. This allows for amplification using a single oligonucleotide in a PCR reaction. For example, if a T7-containing oligonucleotide is used for first strand cDNA synthesis, and a T7 containing oligonucleotide is used for PCR step, then this oligonucleotide can anneal to both the 5′ end and 3′ end of the cDNA. The final PCR product will have T7 RNA polymerase binding sites at both ends. Therefore, in subsequent transcription reactions, the RNA polymerase will bind to both the 5′ and 3′ ends synthesizing the RNA in both sense and antisense orientations.

Transfection of antigen presenting cells with RNA in both the sense and the antisense orientations has produced promising results. However, the negative effects of such procedure on antigen presenting cells has not been previously recognized. Accordingly, methods of optimizing expression of mRNA encoding antigens in dendritic cells and other antigen presenting cells are needed. The present invention satisfies this need.

### SUMMARY

In one embodiment of the presently disclosed subject matter, a method of transfecting an antigen presenting cell with at least one mRNA is provided. The method comprises preparing a preparation essentially devoid of antisense-oriented RNA and double-stranded RNA and comprising at least one sense-oriented mRNA by:
(i) amplifying at least one mRNA from a sample to produce a polynucleotide template, wherein the polynucleotide template comprises a promoter suitable for in vitro transcription operably linked only to a sense strand of the polynucleotide template; and
(ii) in vitro transcribing the polynucleotide template to produce the at least one sense-oriented mRNA, wherein the polynucleotide template is not a cloned template; and
   transfecting at least one antigen presenting cell with the at least one sense-oriented mRNA from the preparation. In some embodiments of the method, amplifying the mRNA from the sample comprises reverse transcribing the mRNA from the sample to produce a polynucleotide template comprising a cDNA; and amplifying the polynucleotide template cDNA using a first primer and a second primer, wherein only one of the first primer and the second primer inserts the promoter suitable for in vitro transcription into the polynucleotide template cDNA. In some embodiments, the first primer comprises a poly T stretch and a 5′ sequence having essentially no sequence homology to the second primer and the second primer comprises the promoter suitable for in vitro transcription.

In another embodiment of the presently disclosed subject matter, an mRNA loaded antigen presenting cell produced by the above method is provided. In some embodiments, the antigen presenting cell is a dendritic cell. Further, in some embodiments, the dendritic cell is a mature dendritic cell. In other embodiments, the dendritic cell is an immature dendritic cell.

In a further embodiment, a composition comprising at least one novel mRNA loaded antigen presenting cell disclosed above in a carrier is provided.

In yet a further embodiment, a method of generating an immune response in a subject against at least one antigen is provided. The method comprises introducing the novel mRNA loaded antigen presenting cell above into a subject, wherein the mRNA loaded antigen presenting cell presents the at least one antigen to the immune system of the subject, thereby generating an immune response against the at least one antigen.

In still a further embodiment, a method of treating a disorder in a subject is provided. The method comprises administering to a subject in need of treatment a therapeutically effective amount of a composition comprising at least one antigen presenting cell transfected in vitro with an RNA preparation comprising at least one sense-oriented mRNA and essentially devoid of antisense-oriented RNA and double-stranded RNA, wherein the at least one sense-oriented mRNA is produced by the methods described above, and in vitro transcribing the polynucleotide template to produce the at least one sense-oriented mRNA, wherein the polynucleotide template is not a cloned template. In some embodiments the disorder is a cancer. In other embodiments, the disorder is a result of a microbial infection. In some embodiments, the antigen presenting cell is an autologous antigen presenting cell obtained from the subject treated.

Accordingly, it is an object of the presently disclosed subject matter to provide a novel method of amplifying RNA to obtain RNA molecules that are predominantly in the sense orientation or in the anti-sense orientation, depending on the desired function. This and other objects are achieved in whole or in part by the presently disclosed subject matter.

An object of the presently disclosed subject matter having been stated hereinabove, other objects and aspects will become evident as the description proceeds when taken in connection with the accompanying Drawings and Examples described herein below.

### BRIEF DESCRIPTION OF THE DRAWINGS

FIGS. 1A and 1B present a schematic representation of the amplification process. FIG. 1A shows the conventional amplification procedure. The schematic indicates that redundant sequences present in the oligonucleotides defining 3′ and 5′ ends of the cDNA could lead to the annealing of T7 containing primer downstream and as a consequence lead to the formation of RNA species transcribed in the antisense orientation. FIG. 1B shows the amplification procedure disclosed herein, which addresses the problem of antisense RNA production.

FIG. 2 is a digital image of an autoradiograph showing a Northern blot analysis performed on total RNA samples (T) and samples amplified using the conventional primers and process (A) using ubiquitin probe complimentary to Sense RNA or Antisense RNA. Equal mass of each RNA (10 μg) was loaded onto each lane. The gel shows antisense RNA for ubiquitin is present in the amplified RNA population.

FIG. 3 is a digital image of an autoradiograph showing a Northern blot analysis of removal of sequence homology between 5′ end and 3′ end primers, which blocks formation of antisense RNA. Northern Blot analysis was performed on samples amplified from SK-Mel 28 RNA or a human tumor RNA. RNA was amplified using the conventional amplification procedure (lanes 1) or using the presently disclosed subject matter (lanes 2). Northern blot analysis was performed using probes recognizing Sense ubiquitin RNA or Antisense ubiquitin RNA.

FIG. 4 shows RNA amplified using the conventional primer (CDS64T) contains double stranded RNAs. Electropheregrams of the RNA samples prior and after digestion with RNAses. Amplified RNA populations (were digested with RNAse specific for single stranded RNA (T1) or additionally with RNAse recognizing double stranded RNA (RNAse III). Left panel shows RNA amplified using the conventional CDS64T oligo and right panel shows RNA amplified using the an CDS64T+ oligo of the presently disclosed subject matter.

FIG. 5 is a dye flip of microarray 1 versus microarray 3. Identical data were obtained in all dye flip comparisons.

FIG. 6 is a scatter flow plot analysis of dendritic cells transfected with different RNAs. Immature dendritic cells (left panel) were transfected with RNA from LNCaP cell line at 2 μg per million cells amplified using conventional CDS 64T oligo (center panel) or novel CDS 64T+ oligo (right panel). D03-017 experiment Gate R1 outlines viable large cells population containing DCs. The number in the lower right corner indicates percent large viable cells present in the sample.

FIG. 7 is a graph showing the phenotype of matured DCs transfected with the two populations of RNA is not different. Immature dendritic cells were harvested on day 6 and electroporated with RNA amplified using: 1) the conventional amplification procedure (dsRNA present); 2) the presently disclosed subject matter (no dsRNA present); or 3) GFP RNA as a control. Cells were placed back in conditioned media to mature overnight and analyzed for phenotypic marker expression: HLA-DR, CD83 and CD14. Y-axis indicates percent positive cells for each analyzed marker.

### DETAILED DESCRIPTION

The practice of the presently disclosed subject matter employs, unless otherwise indicated, conventional techniques of molecular biology (including recombinant techniques), microbiology, cell biology, biochemistry and immunology, which are within the skill of the art. Such techniques are explained fully in the literature. These methods are described in the following publications. See, e.g., Sambrook et al. MOLECULAR CLONING: A LABORATORY MANUAL, 2^{nd} edition (1989); CURRENT PROTOCOLS IN MOLECULAR BIOLOGY (F. M. Ausubel et al. eds. (1987)); the series METHODS IN ENZYMOLOGY (Academic Press, Inc.); PCR: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1988)); USING ANTIBODIES, A LABORATORY MANUAL (Harlow and Lane eds. (1999)); and ANIMAL CELL CULTURE (R. I. Freshney ed. (1987)).

Antigen presenting cells (APCs) play an important role in the treatment of tumors and infectious diseases. APCs process and present antigen on their surface. One way to deliver antigen to an antigen presenting cell is through RNA transfection. Methods of RNA transfection are described in the papers: Boczkowski et al., 2000 and Grunebach et al., 2003. Additional methods of nucleic acid transfection are known to those of skill in the art.

The antigen-encoding RNA can be defined RNA, total RNA or amplified RNA encoding for the antigen(s) of choice. Once the APCs are loaded with RNA, the RNA is translated within the APC, and the resulting protein is processed and presented in the context of an MHC molecule. This initiates an immune response specific to the presented peptides.

RNA can be amplified using a modified version of the CAPswitch technology described in U.S. Pat. Nos. 5,962,271 and 5,962,272 or any other method resulting in a defined sequence at 5′ end. Other methods include, but are not limited to methods involving ligation of defined sequences to 5′ end of RNA such as described in European Patent No. 0 625 572. Each of the above-noted patents is incorporated by reference herein.

Most known methods for amplifying RNA yield an RNA population that includes both sense and anti-sense RNA molecules. However, the prior art has not recognized the detrimental effect that such antisense mRNA can have in the transfection of antigen presenting cells. Transcription of a PCR template in the antisense orientation leads to a lower yield of template in a sense orientation. Furthermore, the antisense species can anneal to sense transcripts leading to sequence-specific double-stranded RNA-mediated gene silencing (Zamore and Aronin, 2003 and Dykxhoorn et al., 2003). These problems are discussed in detail below.

Oppositely oriented transcripts have the potential to form dsRNA sense-antisense duplexes. The number and variety of regulatory phenomena in which dsRNA duplexes are thought to participate is rapidly expanding. For example, dsRNA has been shown to inhibit beta-cell function and induce islet damage by stimulating production of nitric oxide (Blair et al., 2002). Double-stranded RNA has also been shown to be a trigger for apoptosis in vaccinia virus-infected cells (Kibler et al., 1997). Although there has been considerable research on the intermediates involved in dsRNA-triggered cell death, the mechanism of induction of apoptosis by double stranded RNA is not fully characterized.

In addition to the potentially deleterious effects of dsRNA duplexes, anti-sense RNA may play a role in silencing genes important for the viability and function of the antigen presenting cell. The presence of anti-sense RNA also leads to a reduction in the amount of template that is available for the synthesis of functional proteins in cells. In addition, the presence of antisense RNA may promote sequence independent inhibition of protein translation in a cell by RNA dependent protein kinase activated by TLR3.

However, in addition to the effects described above antisense RNA or antisense RNA in the form of double stranded RNA may be used to perform a biological function. One non-limiting example of this is providing maturation stimuli by triggering Toll receptor pathway in DCs.

For the transfection of APCs for the purpose of antigen delivery, a sense strand RNA composition is preferable since sense RNA leads to translation of an antigen encoded by an mRNA. The presence of antisense RNA in the compositions previously used for the transfection of antigen presenting cells has potentially harmful effects. These effects include, but are not limited to (a) reduction of template in a sense orientation for the synthesis of functional protein in a cell; (b) sequence specific silencing of the transduced cells genes via siRNA, antisense RNA or double stranded RNA mechanism and (c) sequence independent inhibition of protein translation in a cell mediated by RNA dependent protein kinase activated by TLR3. The presence of antisense RNA can be detected using various methods such as Northern Blot analysis using a probe specific for a particular antisense RNA. For example, an amplification procedure can be assessed using a probe recognizing ubiquitin antisense RNA.

On the other hand, in certain situations, an anti-sense composition is preferable. For example, it may be desirable to silence a specific gene. In this case it would be desirable to obtain a composition that is essentially devoid of sense-oriented molecules.

The presently disclosed subject matter provides an improved RNA amplification process in which the orientation of the amplified product can be controlled. The method results in a preparation of RNA molecules that are essentially unidirectional. In other words, the RNA will be predominantly in the sense orientation or in the antisense orientation and will be essentially devoid of RNA molecules in the opposite direction and dsRNA, which results from the complementary binding of the oppositely oriented RNA molecules. A preparation of directional RNA in the sense orientation and essentially devoid of molecules in the antisense orientation is provided. As disclosed herein, the antisense-free amplified RNA is a faithful representation of genes expressed by the original cells from which the mRNA is derived, for example, cancer cells. Further, as demonstrated herein in the Examples, transfection of cells with antisense-free RNA results in higher recovery of DCs, which has important implications for large-scale DC-based vaccine manufacturing, and is an improvement over heretofore known procedures.

A preparation of antisense molecules essentially devoid of sense-oriented molecules is also provided.

I. Definitions

“Amplification” refers to nucleic acid amplification procedures using primers and nucleic acid polymerase that generate multiple copies of a target nucleic acid sequence. Such amplification reactions are known to those of skill in the art, and include, but are not limited to, the polymerase chain reaction (PCR, see U.S. Pat. Nos. 4,682,195, 4,683,202 and 4,965,188), RT-PCR (see U.S. Pat. Nos. 5,322,770 and 5,310,652) the ligase chain reaction (LCR, see EP 0 320 308), NASBA or similar reactions such as TMA described in U.S. Pat. No. 5,399,491 and gap LCR (GLCR, see U.S. Pat. No. 5,427,202). If the nucleic acid target is RNA, RNA may first be copied into a complementary DNA strand using a reverse transcriptase (see U.S. Pat. Nos. 5,322,770 and 5,310,652).

“Antigen”, as used herein, refers to a molecule that binds to an antibody or T cell receptor (TCR). The term “antigen” as used herein includes both an entire molecule, and a specific portion (epitope) of the entire molecule that binds to an antibody or TCR. A TCR will bind only fragments of peptides, when complexed with MHC molecules on the surface of APCs, which is referred to as “antigen presentation” by the APC.

“Antigen presenting cells (APCs)” refers to a class of cells capable of presenting one or more antigens in the form of antigen-MHC complex recognizable by specific effector cells of the immune system, and thereby inducing an effective cellular immune response against the antigen or antigens being presented. APCs can be intact whole cells such as macrophages, B-cells, endothelial cells, activated T-cells, and dendritic cells; or other molecules, naturally occurring or synthetic, such as purified MHC Class I molecules complexed to β2-microglobulin. While many types of cells may be capable of presenting antigens on their cell surface for T-cell recognition, only dendritic cells have the capacity to present antigens in an efficient amount to activate naive T-cells for cytotoxic T-lymphocyte (CTL) responses.

By “cancer” is meant the abnormal presence of cells which exhibit relatively autonomous growth, so that a cancer cell exhibits an aberrant growth phenotype characterized by a significant loss of cell proliferation control. Cancerous cells can be benign or malignant. In various embodiments, the cancer affects cells of the bladder, blood, brain, breast, colon, digestive tract, lung, ovaries, pancreas, prostate gland, or skin. The definition of a cancer cell, as used herein, includes not only a primary cancer cell, but also any cell derived from a cancer cell ancestor. This includes metastasized cancer cells, and in vitro cultures and cell lines derived from cancer cells. Cancer includes, but is not limited to, solid tumors, liquid tumors, hematologic malignancies, renal cell cancer, melanoma, breast cancer, prostate cancer, testicular cancer, bladder cancer, ovarian cancer, cervical cancer, stomach cancer, esophageal cancer, pancreatic cancer, lung cancer, neuroblastoma, glioblastoma, retinoblastoma, leukemias, myelomas, lymphomas, hepatoma, adenomas, sarcomas, carcinomas, blastomas, etc. Preferably, the cancer cell is a renal cancer cell, a multiple myeloma cell or a melanoma cell.

“Cloning”, as used herein, means inserting the cDNA made by amplifying RNA according to the methods of the invention into a vector, such as for example, a plasmid vector. The vector usually comprises an origin of replication for amplifying the vector and the cloned polynucleotide sequence of interest. Cloning, as used herein, does not encompass amplification of a polynucleotide sequence of interest generally, and in particular does not encompass polymerase chain reaction amplification. The methods of the presently disclosed subject matter exclude the use of a cloned template for in vitro transcription. Specifically, the methods of the presently disclosed subject matter provide a means to provide essentially sense RNA for the transfection of antigen presenting cells, without the need to clone the template for in vitro transcription.

“Dendritic cells (DCs)” refers to a diverse population of morphologically similar cell types found in a variety of lymphoid and non-lymphoid tissues (Steinman, 1991). Dendritic cells constitute the most potent and preferred APCs in the organism, and provide the signals necessary for T cell activation and proliferation. Dendritic cells are derived from bone marrow progenitor cells, circulate in small numbers in the peripheral blood and appear either as immature Langerhans' cells or terminally differentiated mature cells. Dendritic cells can also be differentiated from monocytes. While the dendritic cells can be differentiated from monocytes, they possess distinct phenotypes. For example, a particular differentiating marker, CD14 antigen, is not found in dendritic cells but is possessed by monocytes. Also, mature dendritic cells are not phagocytic, whereas the monocytes are strongly phagocytosing cells. It has been shown that mature DCs can provide all the signals necessary for T cell activation and proliferation. Methods for the isolation of antigen presenting cells (APCs), and for producing dendritic cell precursors and mature dendritic cells are known to those skilled. See, for example, Berger et al., 2002; U.S. Patent Applications 20030199673 and 20020164346; and WO 93/20185, the contents of which are incorporated by reference. In a preferred embodiment, dendritic cells are prepared from CD14+ peripheral blood monocytic cells (PBMCs) by methods described in Romani et al., 1994 or Sallusto et al., 1994, the contents of which are incorporated by reference. Alternatively, dendritic cells can be prepared from CD34+ cells by the method of Caux et al., 1996.

“Essentially devoid” is used to refer to a preparation in which antisense RNA molecules and dsRNA are present in amounts less than 10%, preferably less than 5%, 4%, 3%, 2%, or 1% of sense mRNA molecules. Preferably, the antisense and dsRNA molecules are undetectable.

“mRNA” means a translatable RNA. The mRNA will contain a ribosome binding site and start codon. Preferably, the mRNA will also contain a 5′ cap, stop codon and polyA tail.

“Operably linked” and “operatively linked”, are used interchangeably herein, and refer to a promoter region that is connected to a nucleotide sequence in such a way that the transcription of that nucleotide sequence is controlled and regulated by that promoter region. Similarly, a nucleotide sequence is said to be under the “transcriptional control” of a promoter to which it is operably linked. Techniques for operatively linking a promoter region to a nucleotide sequence are known in the art.

“Pathogen” refers to any virus or organism which is involved in the etiology of a disease, and also to attenuated derivatives thereof. Such pathogens include, but are not limited to, bacterial, protozoan, fungal and viral pathogens such as Helicobacter, such as Helicobacter pylori, Salmonella sp., Shigella sp., Enterobacter sp., Campylobacter sp., various mycobacteria, such as Mycobacterium leprae, Bacillus anthracis, Yersinia pestis, Francisella tularensis, Brucella sp., Leptospira interrogans, Staphyloccus sp., such as S. aureus, Streptococcus sp., Clostridum sp., Candida albicans, Plasmodium sp., Leishmania sp., Trypanosoma sp., human immunodeficiency virus (HIV), hepatitis C virus (HCV), human papilloma virus (HPV), cytomegalovirus (CMV), human T-cell lymphotropic virus (HTLV), herpesvirus (e.g., herpes simplex virus type 1, herpes simplex virus type 2, coronavirus, varicella-zoster virus, and Epstein-Barr virus), papilloma virus, influenza virus, hepatitis B virus, poliomyelitis virus, measles virus, mumps virus, and rubella virus. The methods of the invention are particularly useful when applied to retroviral pathogens, such as, HIV and HCV.

The pathogen RNA can be in the form of a pathogen genome (e.g., retroviral genomes) or pathogen mRNA, which may be spliced or unspliced. For example, in the case of HIV, HIV RNA could be in the form of a viral RNA genome, isolated from viral particles or from host cells during viral replication, and could also be in the form of HIV mRNA, spliced or unspliced. In a preferred embodiment, the pathogen RNA is HIV genomic RNA isolated from HIV virions. Pathogen RNA can be reverse transcribed to produce a cDNA, which can then serve as a template for nucleic acid amplification.

Preferably, the pathogen RNA is from, or is derived from, multiple pathogen species present in an infected individual. In this context, by “derived from” is meant that the nucleic acid is at least partially purified from the pathogen or cell(s) containing pathogen nucleic acid, or that the nucleic acid is amplified from pathogen nucleic acid. By deriving the nucleic acid from multiple pathogen species present in an individual, the resulting vaccine can elicit an immune response to potentially all of the species of pathogen present in an individual.

II. Methods of Amplification and In Vitro Transcription of mRNA

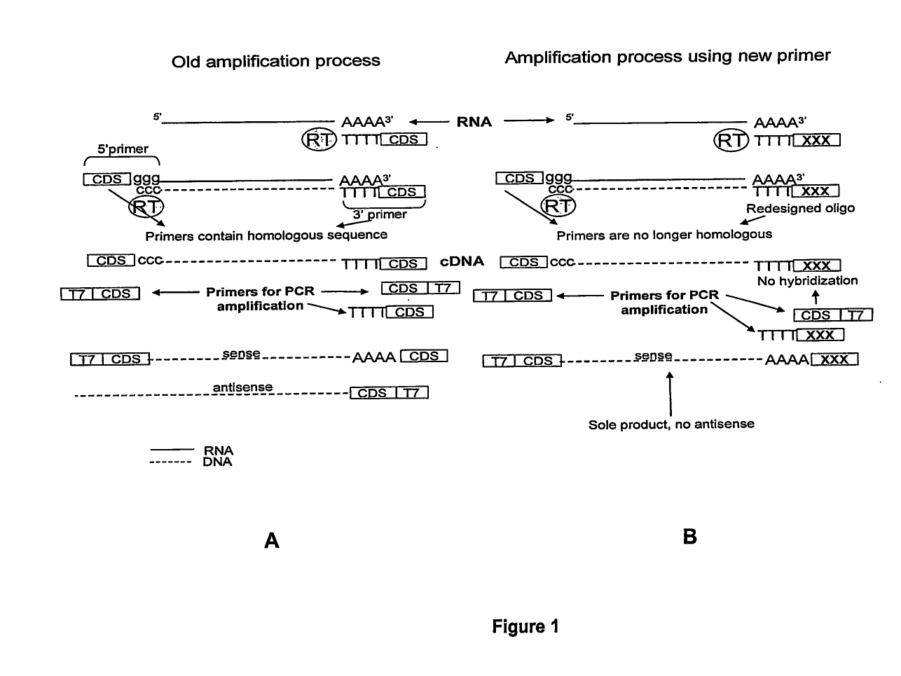

Representative advantages of the subject matter disclosed herein can be seen in a generally diagrammatical comparison of a novel method disclosed herein to a standard amplification method. In FIG. 1A, the steps involved in a standard amplification procedure are illustrated schematically. The steps involved in one example of the novel methods of the presently disclosed subject matter are shown in FIG. 1B.

Referring to FIG. 1A, the standard RNA amplification, is a modified version of a SMART amplification procedure (Clontech) which provides defined 3′ and 5′ ends of first strand cDNA in one RT reaction due to the unique property of a mutant RT (Powerscript Reverse Transcriptase, Clontech) used in the procedure. An oligonucleotide that is linked to the 5′ end of the mRNA serves as a short extended template so that when the reverse transcriptase enzyme reaches the 5′ end of the mRNA, the enzyme switches templates and proceeds through the end of linked oligonucleotide. Thus the 5′ end of the cDNA has a defined sequence termed a complimentary defined sequence or CDS.

As shown in FIG. 1A, the 3′ end of the cDNA is defined by supplying an oligo containing a defined sequence (CDS) as well as a poly T stretch. The polyT portion of that oligo anneals to poly A region of mRNA and serves as an anchor for first strand cDNA synthesis. In this manner, the 3′ end of cDNA contains a CDS defined sequence. The presence of the defined oligonucleotide sequence at both ends of cDNA makes it possible to amplify cDNA in a PCR reaction. PCR primers are provided as follows: TTTT[CDS] as a 3′ primer and T7[CDS] as a 5′ primer. The T7 portion of the 5′ primer is a T7 RNA polymerase binding site which allows for production of a polynucleotide template comprising a T7 promoter, which facilitates RNA transcription of the polynucleotide template at the final step. In this standard protocol shown in FIG. 1A, the original 3′ primer and the 5′ primer share common sequences and thus the T7[CDS] primer is able to anneal to both ends. As a result, a polynucleotide template, for example a cDNA, containing T7[CDS] sequence is synthesized in both orientations and this ultimately leads to the formation of RNA in both orientations.

In this amplification procedure, the desired product is RNA, not DNA. Thus, an RNA polymerase binding sequence is incorporated on both strands of a polynucleotide template during the PCR step. When the RNA polymerase binding sequence is annealed to the 3′ end of the polynucleotide template cDNA, antisense RNA is produced and when the RNA polymerase binding domain is annealed at the 5′ end of the polynucleotide template cDNA, sense RNA is produced. Thus, the final product contains RNA oriented in both directions. As discussed above, the presence of both sense and anti-sense RNA in the product can result in the generation of dsRNA which can have deleterious effects on cells transfected with the RNA product.

RNA orientation or direction is defined as either one that encodes the gene of interest (sense orientation) or opposite to the sense orientation (antisense orientation)

The presently disclosed subject matter provides several advantages over the prior art methods. The presence of dsRNA is eliminated by providing methods which produce only sense or only anti-sense RNA. The compositions of the subject matter disclosed herein are superior to composition produced by other methods since they are essentially devoid of RNA in the opposite orientation. This is achieved by using primers that essentially have no sequence homology to each other. One of the primers inserts an RNA polymerase binding site. Depending on whether the RNA polymerase binding site is inserted at the 5′ or the 3′ end, only sense or only anti-sense RNA will be produced when the cDNA is transcribed into RNA.

II.A. Methods for Producing Sense-Oriented RNA Essentially Devoid of Antisense RNA and dsRNA

In one aspect of the presently disclosed subject matter, the formation of antisense RNA is blocked. The starting RNA to be amplified is typically polyA+ RNA, which can be isolated by using conventional methods (e.g., use of poly dT chromatography). Both cytoplasmic and nuclear RNA, either together or separated, are useful in the subject matter disclosed herein. Also useful in the presently disclosed subject matter is RNA corresponding to tumor or pathogen antigens or epitopes unique to the tumor or pathogen, and RNA corresponding to “minigenes” (i.e., RNA sequences encoding defined epitopes). Tumor-specific or pathogen-specific RNA can be derived by separation of the unique antigens from the total RNA population by any of a number of methods generally known in the art. As one non-limiting example, subtractive hybridization methods can be utilized to separate unique RNA from a total RNA population. Methods for subtractive hybridization are known to those skilled in the art. See, for example, U.S. Pat. Nos. 5,256,536 and 6,800,734 and Utt et al., 1995, the contents of which are incorporated by reference. As a nonlimiting example, total mRNA from a cancer cell (e.g., renal cell cancer) can be used as substrate to prepare a set of cDNA molecules corresponding to all the expressed genes. To remove sequences that are not specific for (or preferentially expressed in) the target cells, the cDNA preparation is exhaustively hybridized with the mRNA of corresponding normal cells (e.g., normal renal tissue). This step removes all the sequences from the cDNA preparation that are common to the two cell types. After discarding all the cDNA sequences that hybridize with the other mRNA, those that are left comprise a selected fraction of the total mRNA population and contain sequences unique to the mRNA population of the cancer cell.

FIG. 1B illustrates one embodiment of the presently disclosed subject matter. In this illustration, T7 polymerase is used, however any RNA polymerase suitable for in vitro transcription and its corresponding binding site in a context oligonucleotides for PCR will apply as well. It is clearly apparent that different primers and different polymerases could be used to achieve the same result. It is also clearly apparent that the depicted method can be modified to produce only anti-sense molecules. The presently disclosed subject matter encompasses a generalized scheme for the preferential production of RNA in one orientation.

In a novel method disclosed herein shown in FIG. 1B, the generation of anti-sense RNA is blocked. This is achieved by redesigning the original polyT containing primer to contain a unique sequence that has no homology to the 5′ primer. This leads to the formation of a polynucleotide template, preferably a cDNA, containing distinct and unique ends that can be transcribed by a specific polymerase, for example T7 polymerase. The T7[CDS] primer anneals to the 5′ end only and not to the 3′ end, and thus no antisense cDNA containing the T7 promoter is synthesized. It therefore follows that RNA synthesis in the antisense orientation will be affected and an RNA preparation will be synthesised which is essentially devoid of RNA molecules in the antisense orientation.

The above-described method generates a preparation of RNA molecules which is essentially devoid of antisense RNA, that is, there is virtually no antisense RNA present in the preparation. The preparation of the presently disclosed subject matter has the advantageous feature that it is less toxic to antigen presenting cells than other RNA preparations. Antigen presenting cells transfected with this preparation result in a higher yield of healthy cells. Without being limited by theory, the surprising effects on transfected cells may be because the deleterious effects of the antisense orientation or dsRNA are eliminated. This results in a yield of transfected cells that is enhanced both quantitatively and qualitatively. Cells transfected with only sense RNA exhibit higher viability and the number of doses obtained is greater as compared to cell populations transfected with both sense and antisense RNA. Since RNA electroporated DC vaccine manufacturing is relatively expensive, the increased number of doses obtained has a significant economic benefit. The increase in the number of doses obtained also has health benefits for the patient to be treated.

II.B. Methods to Detect Nucleic Acids

Various methods are known for quantifying sense, antisense and dsRNA and include, but are not limited to, hybridization assays (Northern blot analysis) and PCR based hybridization assays.

mRNA can be isolated using various lytic enzymes or chemical solutions according to the procedures set forth in Sambrook et al., 1989, or extracted by commercially available nucleic-acid-binding resins following the accompanying instructions provided by the manufacturers. The mRNA contained in the extracted nucleic acid sample is then detected by hybridization (e.g., Northern blot analysis) and/or amplification procedures using nucleic acid probes and/or primers, respectively, according to standard procedures.

Nucleic acid molecules having at least 10 nucleotides and exhibiting sequence complementarity or homology to the nucleic acid to be detected can be used as hybridization probes or primers in the diagnostic methods. It is known in the art that a “perfectly matched” probe is not needed for a specific hybridization. Minor changes in probe sequence achieved by substitution, deletion or insertion of a small number of bases do not affect the hybridization specificity. In general, as much as 20% base-pair mismatch (when optimally aligned) can be tolerated. The total size of fragment, as well as the size of the complementary stretches, will depend on the intended use or application of the particular nucleic acid segment. Smaller fragments of the gene will generally find use in hybridization embodiments, wherein the length of the complementary region may be varied, such as between about 10 and about 100 nucleotides, or even full length according to the complementary sequences one wishes to detect.

Nucleotide probes having complementary sequences over stretches greater than about 10 nucleotides in length will increase stability and selectivity of the hybrid, thereby improving the specificity of particular hybrid molecules obtained. One can design nucleic acid molecules having gene-complementary stretches of more than about 25 and even more preferably more than about 50 nucleotides in length, or even longer where desired. Such fragments may be readily prepared by, for example, directly synthesizing the fragment by chemical means, by application of nucleic acid reproduction technology, such as the PCR™ technology with two priming oligonucleotides as described in U.S. Pat. No. 4,603,102, herein incorporated by reference, or by introducing selected sequences into recombinant vectors for recombinant production.

In certain embodiments, it will be advantageous to employ nucleic acid sequences of the presently disclosed subject matter in combination with an appropriate means, such as a label, for detecting hybridization and therefore complementary sequences. A wide variety of appropriate indicator means are known in the art, including fluorescent, radioactive, enzymatic or other ligands, such as avidin/biotin, which are capable of giving a detectable signal. A fluorescent label or an enzyme tag, such as urease, alkaline phosphatase or peroxidase, instead of radioactive or other environmental undesirable reagents can also be used. In the case of enzyme tags, colorimetric indicator substrates are known which can be employed to provide a means visible to the human eye or spectrophotometrically, to identify specific hybridization with complementary nucleic acid-containing samples.

Hybridization reactions can be performed under conditions of different “stringency”. Relevant conditions include temperature, ionic strength, time of incubation, the presence of additional solutes in the reaction mixture such as formamide, and the washing procedure. Higher stringency conditions are those conditions, such as higher temperature and lower sodium ion concentration, which require higher minimum complementarity between hybridizing elements for a stable hybridization complex to form. Conditions that increase the stringency of a hybridization reaction are widely known and published in the art. See, Sambrook et al., 1989.

One can also detect and quantify mRNA level or its expression using quantitative PCR or high throughput analysis such as Serial Analysis of Gene Expression (SAGE) as described in Velculescu et al., 1995. Briefly, the method comprises isolating multiple mRNAs from cell or tissue samples suspected of containing the transcript. Optionally, the gene transcripts can be converted to cDNA. A sampling of the gene transcripts are subjected to sequence-specific analysis and quantified. These gene transcript sequence abundances are compared against reference database sequence abundances including normal data sets for diseased and healthy patients. The patient has the disease(s) with which the patient's data set most closely correlates and for this application, includes the differential of the transcript.

In certain aspects, it may be necessary to use polynucleotides as nucleotide probes or primers for the amplification and/or detection of RNA. A primer useful for detecting differentially expressed mRNA is at least about 80% identical to the homologous region of comparable size of a gene or polynucleotide. For the purpose of the subject matter disclosed herein, amplification means any method employing a primer-dependent polymerase capable of replicating a target sequence with reasonable fidelity. Amplification may be carried out by natural or recombinant DNA-polymerases such as T7 DNA polymerase, Klenow fragment of E. coli DNA polymerase, and reverse transcriptase.

General procedures for PCR are taught in MacPherson et al., 1991. However, PCR conditions used for each application reaction are empirically determined. A number of parameters influence the success of a reaction. Among them are annealing temperature and time, extension time, Mg²⁺ ATP concentration, pH, and the relative concentration of primers, templates, and deoxyribonucleotides.

After amplification, the resulting DNA fragments can be detected by agarose gel electrophoresis followed by visualization with ethidium bromide staining and ultraviolet illumination. A specific amplification of differentially expressed genes of interest can be verified by demonstrating that the amplified DNA fragment has the predicted size, exhibits the predicated restriction digestion pattern, and/or hybridizes to the correct cloned DNA sequence. Other methods for detecting gene expression are known to those skilled in the art. See, for example, PCT Application No. WO 97/10365; U.S. Pat. Nos. 5,405,783; 5,412,087; 5,445,934; 5,405,783; 5,412,087; 5,445,934; 5,578,832; and 5,631,734; and Tijssen (ed.), 1993.

III. Methods of Transfecting APCs

The presently disclosed subject matter provides a composition of APCs transfected with sense-oriented RNA. Methods of culturing and transfecting antigen presenting cells are generally known. Exemplary methods are disclosed in pending U.S. Provisional Patent Application No. 60/522,512, the contents of which are herein incorporated by reference. However, the method of transfection is not critical to the invention.

III.A. Isolating and Culturing APCs

Methods for isolating and culturing antigen presenting cells are known to those of skill in the art. As a nonlimiting example, immature DCs can be isolated or prepared from a suitable tissue source containing DC precursor cells and differentiated in vitro to produce immature DCs. For example, a suitable tissue source can be one or more of bone marrow cells, peripheral blood progenitor cells (PBPCs), peripheral blood stem cells (PBSCs), and cord blood cells. In one embodiment, the tissue source is a peripheral blood mononuclear cell (PBMC). The tissue source can be fresh or frozen. In another aspect, the cells or tissue source are pre-treated with an effective amount of a growth factor that promotes growth and differentiation of non-stem or progenitor cells, which are then more easily separated from the cells of interest. These methods are known in the art and described in Romani et al., 1994 and Caux, C. et al., 1996.

Antigen presenting cells (APCs) include, but are not limited to, macrophages, endothelial cells, B-cells and dendritic cells, such as immature dendritic cells, mature dendritic cells and Langerhans cells. Professional antigen-presenting cells, in particular dendritic cells (DCs), provide a powerful vehicle for stimulation of cell-mediated immunity through effects on both CD4+ and CD8+T cells (Banchereau, 1998 and Banchereau, 2000). Intrinsic to their function, they efficiently process antigens for presentation on both MHC class I and II products to CD4+ and CD8+T cells. Dendritic cells, isolated from peripheral blood or bone marrow through surface antigen enrichment techniques or harvested from cultures of PBMCs, can be loaded with specific candidate antigens and are then capable of presenting the relevant antigen(s) to naïve or resting T cells (Heiser, 2000 and Mitchell, 2000). Preferably, the antigen presenting cells are autologous to the individual to be vaccinated, and the pathogen or cancer cell RNA is isolated or derived from the patient as well.

III.A.1. Isolating APCs from PBMCs

In one aspect, the immature DCs are isolated from peripheral blood mononuclear cells (PBMCs). In a preferred embodiment, the PBMCs are treated with an effective amount of granulocyte macrophage colony stimulating factor (GM-CSF) in the presence or absence of interleukin 4 (IL4) and/or IL-13, so that the PBMCs differentiate into immature DCs. Most preferably, PBMCs are cultured in the presence of GM-CSF and IL-4 for approximately six days to produce immature DCs, suitable for use in the methods of the presently disclosed subject matter.

III.A.2. Isolating APCs from Stem Cells

Many methods are known in the art for the isolation of stem cells for in vitro expansion and differentiation. The following descriptions are for the purpose of illustration only and in no way are intended to limit the scope of the presently disclosed subject matter.

Stem cells can be isolated from bone marrow cells by panning the bone marrow cells with antibodies which bind unwanted cells, such as CD4+ and CD8⁺ (T cells), CD45⁺ (panB cells) and GR-1 For a detailed description of this protocol see, Inaba et al., 1992.

Human CD34⁺ cells can be obtained from a variety of sources, including cord blood, bone marrow, and mobilized peripheral blood. Purification of CD34⁺ cells can be accomplished by antibody affinity procedures. See, for example, Paczesny et al., 2004; Ho et al., 1995; Brenner, 1993; and Yu et al., 1995.

DCs can also be generated from frequent, but non-proliferating CD14⁺ precursors (monocytes) in peripheral blood under the aegis of GM-CSF+IL-4 (see, e.g., WO 97/29182). This method is described in Sallusto and Lanzavecchia, 1994 and Romani et al., 1994. Briefly, CD14⁺ precursors are abundant so that pretreatment of patients with cytokines such as G-CSF (used to increase CD34⁺ cells and more committed precursors in peripheral blood) is reported to be unnecessary in most cases. See Romani et al., 1996. Others have reported that DCs generated by this approach appear rather homogenous and can be produced in an immature state or fully differentiated or mature. It was shown that it is possible to avoid non-human proteins such as FCS (fetal calf serum), and to obtain fully and irreversibly mature and stable DC by using autologous monocyte conditioned medium as maturation stimulus. Romani et al., 1996; Bender et al., 1996. However, in contrast to the presently disclosed subject matter, these studies did not result in mature DCs having increased levels of IL-12 and/or decreased levels of IL-10.

Stem cells can be differentiated into dendritic cells by incubating the cells with the appropriate cytokines. Inaba et al., 1994, described the in vitro differentiation of murine stem cells into dendritic cells by incubating the stem cells with murine GM-CSF. In brief, isolated stem cells are incubated with between 1 and 200 ng/ml murine GM-CSF, and preferably about 20 ng/ml GM-CSF in standard RPMI growth medium. The media is changed with fresh media about once every other day. After 7 days in culture, a large percentage of cells are dendritic, as assessed by expression of surface markers and morphology. Dendritic cells are isolated by florescence activated cell sorting (FACS) or by other standard methods.

Immature dendritic cells can be prepared from CD34⁺ hematopoietic stem or progenitor cells. The CD34⁺ hematopoietic stem or progenitor cells can be isolated from a tissue source selected from the group consisting of bone marrow cells, peripheral blood progenitor cells (PBPCs), peripheral blood stem cells (PBSCs), and cord blood cells. Human cells CD34⁺ hematopoietic stem cells are preferably differentiated in vitro by culturing the cells with human GM-CSF and TNF-α. See for example, Szabolcs et al., 1995.

For mouse DCs, murine stem cells can be differentiated into dendritic cells by incubating the stem cells in culture with murine GM-CSF. Typically, the concentration of GM-CSF in culture is at least about 0.2 ng/ml, and preferably at least about 1 ng/ml. Often the range will be between about 20 ng/ml and 200 ng/ml. In many preferred embodiments, the dose will be about 100 ng/ml. IL4 is optionally added in similar ranges for making murine DCs.

When human cells are transduced, human GM-CSF is used in similar ranges, and TNF-α also is added to facilitate differentiation. TNF-α is also typically added in about the same ranges. Optionally, SCF or other proliferation ligand (e.g., Flt3) is added in similar dose ranges to make human DCs.

As is apparent to those of skill in the art, all of the above-noted dose ranges for differentiating stem cells are approximate. Different suppliers and different lots of cytokine from the same supplier vary in the activity of the cytokine. One of skill can easily titrate each cytokine, which is used to determine the optimal dose for any particular cytokine.

III.B. Transfecting APCs

A novel method of the presently disclosed subject matter can be used to provide RNA transfected antigen presenting cells for a variety of therapeutic applications where it is desirable to induce or modulate an immune response to a presented peptide. In one embodiment of the presently disclosed subject matter, a preparation of sense oriented RNA derived from tumor cells is used to transfect antigen presenting cells to provide a composition of cells that is useful as a cancer immunotherapeutic. In another embodiment of the presently disclosed subject matter, a preparation of sense oriented RNA derived from one or more pathogens can be used to transfect cells and provide a composition useful in the treatment and/or prevention of infectious diseases. Further, in another embodiment of the presently disclosed subject matter, sense oriented RNA encoding tolerogenic peptides can also be used in the preparation of APCs useful in the treatment of autoimmune disease.

Methods for transfecting antigen presenting cells are known to those of skill in the art, and include, but are not limited to, electroporation, passive uptake, lipofection, cationic reagents, viral transduction, CaPO4, nanoparticle-mediated transfection, peptide-mediated transfection and the like. For example, methods for peptide mediated transfection are disclosed in U.S. Patent Application Nos. 20030125242 and 20030087810, the contents of which are incorporated by reference. Other methods of peptide mediated transfection are known to those of skill in the art. Preferably, the peptide is a cationic peptide. Methods for nanoparticle-mediated transfection are also known to those of skill in the art. See, for example, U.S. Patent Application No. 20040038406, the contents of which are incorporated by reference. The method of transfection is not critical to the presently disclosed subject matter and therefore the preceding list of methods is intended to be exemplary, and in no way is it intended to limit the scope of the presently disclosed subject matter.

Dendritic cells can be loaded in vitro when mature or immature, and then matured in vitro prior to vaccination or in vivo following vaccination. Alternatively, nucleic acids can be delivered to antigen presenting cells in situ. Methods of in situ transfection are known to those skilled in the art. See, for example, U.S. Patent Application Nos. 20040082530 and 20030032615, PCT Application No. WO 01/23537, U.S. Pat. No. 6,603,998, Hoerr et al., 2000, Liu et al., 2002; Lisziewics et al., 2003; and O'Hagen, 2001, the contents of which are incorporated by reference.

Preferably, the APC transfected is a professional APC such as a dendritic cell or a macrophage. Alternatively, artificially generated APCs can be used. The APC can be transfected with the sense RNA using conventional methods. For example, an APC may be contacted with the tumor-derived RNA in the presence of a cationic lipid. Other known transfection methods can also be used to introduce the RNA into the APC. U.S. Pat. No. 6,306,388 discloses methods for transfecting APCs with RNA. RNA derived from essentially any type of cancer cell or pathogen can be used as the starting material for the amplification method. In other situations, it may be desirable to amplify RNA encoding tolerogenic proteins.

IV. In Vivo Therapy

T cells or dendritic cells produced by the methods of the presently disclosed subject matter can be administered directly to the subject to produce T cells active against a selected immunogen. Administration can be by methods known in the art to successfully deliver a cell into ultimate contact with a subject's blood or tissue cells.

The cells are administered in any suitable manner, often with pharmaceutically acceptable carriers. Suitable methods of administering cells in the context of the presently disclosed subject matter to a subject are available, and, although more than one route can be used to administer a particular cell composition, a particular route can often provide a more immediate and more effective reaction than another route.

Pharmaceutically acceptable carriers are determined in part by the particular composition being administered, as well as by the particular method used to administer the composition. Accordingly, there are a wide variety of suitable formulations of pharmaceutical compositions of the presently disclosed subject matter. Most typically, quality controls (microbiology, clonogenic assays, viability tests), are performed and the cells are reinfused back to the subject, preceded by the administration of diphenhydramine and hydrocortisone. See, for example, Korbling et al., 1986 and Haas et al., 1990.

Formulations suitable for parenteral administration, such as, for example, by intraarticular (in the joints), intravenous, intramuscular, intradermal, intraperitoneal, and subcutaneous routes, and carriers include aqueous isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Intravenous or intraperitoneal administration are the preferred method of administration for dendritic or T cells of the presently disclosed subject matter.

The dose of cells (e.g., activated T cells, or dendritic cells) administered to a subject is in an effective amount, effective to achieve the desired beneficial therapeutic response in the subject over time, or to inhibit growth of cancer cells, or to inhibit infection.

For the purpose of illustration only, the method can be practiced by obtaining and saving blood samples from the subject prior to infusion for subsequent analysis and comparison. Generally at least about 10⁴ to 10⁶ and typically, between 1×10⁸ and 1×10¹⁰ cells are infused intravenously or intraperitoneally into a 70 kg patient over roughly 60-120 minutes. In one aspect, administration is by intravenous infusion. Vital signs and oxygen saturation by pulse oximetry are closely monitored. Blood samples are obtained 5 minutes and 1 hour following infusion and saved for analysis. Cell re-infusions are repeated roughly every month for a total of 10-12 treatments in a one year period. After the first treatment, infusions can be performed on an outpatient basis at the discretion of the clinician. If the re-infusion is given as an outpatient, the participant is monitored for at least 4 hours following the therapy.

For administration, cells of the presently disclosed subject matter can be administered at a rate determined by the LD-50 (or other measure of toxicity) of the cell type, and the side effects of the cell type at various concentrations, as applied to the mass and overall health of the subject. Administration can be accomplished via single or divided doses. The cells of the subject matter disclosed herein can supplement other treatments for a condition by known conventional therapy, including cytotoxic agents, nucleotide analogues and biologic response modifiers. Similarly, biological response modifiers are optionally added for treatment by the DCs or activated T cells of the presently disclosed subject matter. For example, the cells are optionally administered with an adjuvant, or cytokine such as GM-CSF, IL-12 or IL-2.

V. Methods to Assess Immunogenicity

The immunogenicity of the antigen presenting cells or educated T cells produced by the methods of the presently disclosed subject matter can be determined by well known methodologies including, but not limited to the following:

⁵¹Cr-release lysis assay. Lysis of peptide-pulsed ⁵¹Cr-labeled targets by antigen-specific T cells can be compared. “More active” compositions will show greater lysis of targets as a function of time. The kinetics of lysis as well as overall target lysis at a fixed timepoint (e.g., 4 hours) may be used to evaluate performance. Ware et al., 1983.

Cytokine-release assay. Analysis of the types and quantities of cytokines secreted by T cells upon contacting modified APCs can be a measure of functional activity. Cytokines can be measured by ELISA or ELISPOT assays to determine the rate and total amount of cytokine production. Fujihashi et al., 1993; Tanquay and Killion, 1994.

In vitro T cell education. The compositions of the presently disclosed subject matter can be assayed for the ability to elicit reactive T cell populations from normal donor or patient-derived PBMC. In this system, elicited T cells can be tested for lytic activity, cytokine-release, polyclonality, and cross-reactivity to the antigenic epitope. Parkhurst et al., 1996.

Transgenic animal models. Immunogenicity can be assessed in vivo by vaccinating HLA transgenic mice with the compositions of the presently disclosed subject matter and determining the nature and magnitude of the induced immune response. Alternatively, the hu-PBL-SCID mouse model allows reconstitution of a human immune system in a mouse by adoptive transfer of human PBL. These animals may be vaccinated with the compositions and analyzed for immune response as previously mentioned in Shirai et al., 1995; Mosier et al., 1993.

Proliferation Assays. T cells will proliferate in response to reactive compositions. Proliferation can be monitored quantitatively by measuring, for example, ³H-thymidine uptake. Caruso et al., 1997.

Primate models. A non-human primate (chimpanzee) model system can be utilized to monitor in vivo immunogenicities of HLA-restricted ligands. It has been demonstrated that chimpanzees share overlapping MHC-ligand specificities with human MHC molecules thus allowing one to test HLA-restricted ligands for relative in vivo immunogenicity. Bertoni et al., 1998.

Monitoring TCR Signal Transduction Events. Several intracellular signal transduction events (e.g., phosphorylation) are associated with successful TCR engagement by MHC-ligand complexes. The qualitative and quantitative analysis of these events has been correlated with the relative abilities of compositions to activate effector cells through TCR engagement. Salazar et al., 2000; Isakov et al., 1995.

VI. Vaccines and Methods of Using

The presently disclosed subject matter further provides a vaccine composition comprising the loaded antigen presenting cells described above. In such vaccines, the loaded antigen presenting cells will be in a buffer suitable for therapeutic administration to a subject. The vaccine may further comprise an adjuvant for factors for the stimulation of antigen presenting cells or T cells. Methods of formulating pharmaceutical compositions are known to those skilled in the art. See, for example, the latest version of Remington's Pharmaceutical Science.

The optimal immunization interval for dendritic cell vaccines can be determined by one of skill in the art. In a preferred embodiment, subjects will be vaccinated 5 times with between 1×10⁶ to 1×10⁷ viable RNA-loaded DCs per dose. The dose level selected for vaccination is expected to be safe and well-tolerated.

Methods of isolating, preparing, transfecting, formulating and administering antigen presenting cells to patients are known in the art. See, for example, Fay et al, 2000; Fong et al., 2001; Ribas et al., 2001; Schuler-Thurneret al., 2002; and Stift et al., 2003, the contents of which are incorporated by reference.

Routes of APC administration employed clinically include, but are not limited to, intravenous (IV), subcutaneous (SC), intradermal (ID), and intralymphatic. Objective clinical responses have been reported following IV, SC, and ID dosing. Currently, there is a developing preference for ID administration since the dermis is a normal residence for dendritic cells from which they are known to migrate to draining lymph nodes. In murine models, SC-injected dendritic cells are later found in T cell areas of draining lymph nodes and trigger protective antitumor immunity that is superior to that following IV immunization. There is murine evidence that dendritic cell injection directly into a lymph node is superior to other routes of delivery in generating protective antitumor immunity or cytotoxic T-lymphocytes (CTLs) (Lambert et al., 2001, the contents of which are incorporated by reference). This suggests that an entire dendritic cell dose should be delivered so that it impacts on a single draining lymph node or basin (rather than dividing the dose among multiple sites to engage as many nodes as possible)

To assess the immunogenicity of the vaccine, immune responses in vaccinated individuals can be monitored by following the maturation profiles of CD4+ and CD8+T cells. For example, cancer cell-specific or pathogen-specific effector cell function can be determined by the presence of cells expressing the phenotype of effector T-cells, CD45 RA+ CCR7- and secreting elevated levels of γ-IFN and granzyme B. Restoration of HIV-specific proliferative responses can be determined by the capacity of cells to produce II-2 and to become CFSE low following stimulation with dendritic cells transfected with HIV—RNAs Restoration HIV-specific memory T cell compartment.

Maturation of specific T cells induced by the vaccine can be measured using surface and intracellular markers using flow cytometry assay. CD8+ T cells will be monitored by staining for surface markers including TCR, CD45RA, CCR7 and CD107 or intracellular molecules such as granzyme β or γ-IFN. CD3, CD4,CCR7 and IL-2 can be used to monitor CD4+T cells. Such assays can be used to monitor immune response following incubation with peptides encompassing the autologous HIV sequences from the patient. Comparison of the cellular immune responses at baseline and monthly prior to each new vaccination allows determination the impact of the vaccine on the breadth of the cellular immune response. The breadth of the immune response can also be measured using the CFSE proliferation assay.

VII. Additional Applications

The presently disclosed subject matter is not limited to antigen delivery to antigen presenting cells. The presently disclosed subject matter can be applied to the delivery of defined targets to any cell line or tissue cells. The RNA of the presently disclosed subject matter can also be used to transfect other cell types to provide a template for the synthesis of a functional protein in a cell.

Any process, which is based on the use of amplified RNA or cDNA, in which preferential amplification of one orientation is desired will benefit from the novel methods disclosed herein.

The presently disclosed subject matter can also be applied to the production of defined antigen targets if those targets are PCR amplified from a particular source.

The methods, RNA preparations and cell compositions of the presently disclosed subject matter provide significant advantages over the prior art. The presently disclosed subject matter provides a highly efficient method for the production of qualitatively superior RNA preparations and their use in cell compositions.

It is clearly apparent that while the methodology has been described in detail more fully for the preferential amplification of sense RNA, the same concept can be applied in the other orientation to preferentially amplify anti-sense RNA. Transfection with anti-sense RNA also has many therapeutic and research purposes.

The above disclosure generally describes the presently disclosed subject matter. A more complete understanding can be obtained by reference to the following specific examples. These examples are described solely for purposes of illustration and are not intended to limit the scope of the presently disclosed subject matter. Changes in form and substitution of equivalence are contemplated as circumstances may suggest or render expedient. Although specific terms have been employed herein, such terms are intended in a descriptive sense and not for purposes of limitation.

### EXAMPLES

### Example 1

### RNA Amplification Using Novel Oligonucleotides Compared to Conventional Oligonucleotides

A. Oligonucleotides Used for RNA Amplification

Conventional(CDS64T)

(SEQ ID NO: 1)

5′-AAGCAGTGGTAACAACGCAGAGTAC(T)₆₃VN-3′
Novel(CDS64T + oligo)

(SEQ ID NO: 2)

5′-CGATAAAAGCTCCGGGGATAACAGA(T)₆₃VN-3′
V = A, G, or C
N = A, G, C, or T
Capswitch

(SEQ ID NO: 3)

5′-AAGCAGTGGTAACAACGCAGAGTACGCGGG-3′
T7 capswitch

(SEQ ID NO: 4)

5′-TAATACGACTCACTATAGGGAGGAAGCAGTGGTAACAACGCAGAGT-
3′

B. RNA Amplification

One microgram of total RNA from an SKMel 28 cell line or renal cell carcinoma tumor specimen was used in a 10 μL reverse transcriptase reaction containing 1 μM CapSwitch primer, 1 μM of CDS64T or CDS 64 T+ oligo primer, 1 μL POWERSCRIP™ reverse transcriptase (BD Biosciences Clontech, Palo Alto, Calif., U.S.A.), 1× first strand synthesis buffer, 1 μM dNTPs, 2 mM DTT. The reaction mixture was incubated for 1 hr at 42° C.

2 μL of the RT product was then diluted into a 100 μL PCR reaction mixture containing 0.4 μM f T7 Capswitch and CDS64T or CDS 64T+ oligo primers, 0.4 μM dNTPs, 1× KlenTaq PCR buffer and 1 μL Advantage KlenTaq polymerase mix (BD Biosciences Clontech). Amplification was achieved following 20 cycles 95° C. 5 sec, 65° C. 5 sec, 68° C. 6 min. The amplified cDNA was purified using a PCR purification kit (QIAGEN, Valencia, Calif., U.S.A.). 3 μg of each cDNA was transcribed in in vitro transcription reaction using a T7 MMACHINE MMESSAGE® kit (Ambion, Woodward, Tex., U.S.A.) according to manufacturer's instructions. Final RNA was purified using a RNeasy mini column (QIAGEN) following the protocol for RNA cleanup.

### Example 2

### Determination of Antisense RNA Presence in the Amplified RNA Population Using Conventional Oligonucleotides

A. Northern Blot Analysis

RNA was resolved on 1.2% denaturing agarose gel and transferred onto Nylon Membrane using capillary transfer in 10×SSC. After overnight transfer the RNA was cross-linked to the membrane. Overnight hybridization was performed in EXPRESSHYB™ Solution (BD Biosciences Clontech, Palo Alto, Calif., U.S.A.) at 68° C. After hybridization, the membrane washed with low stringency wash 2×SSC, 0.5% SDS at room temperature and high stringency wash 1×SSC, 0.1% SDS at 55° C. and exposed to Phosphoimager screen.

The single-stranded probes used to detect RNAs in the sense and antisense directions were designed to be complementary to ubiquitin NCBI mRNA sequence of human ubiquitin mRNA, GenBank accession #M26880

Ubiquitin Sense Probe

(SEQ ID NO: 5)

5′-GAGAACGTCAAGGCAAAGATCCAGGACAAGGAAGGCATTCCTCCTGA
CCAGCAGAGGTTGATCTTTGCCGGAAAGCAGCTGGAAGATGGGCGGACCC
TGT-3′
Ubiquitin Antisense Probe

(SEQ ID NO: 6)

5′-ACAGGGTCCGCCCATCTTCCAGCTGCTTTCCGGCAAAGATCAACCTC
TGCTGGTCAGGAGGAATGCCTTCCTTGTCCTGGATCTTTGCCTTGACGTT
CTC-3′

Labelling of all double-stranded probes was performed using Deka Prime II kit (Ambion, Woodward, Tex., U.S.A.) using manufacturer's instructions. Labelling of all single-stranded probes were performed using the KINASEMAX™ End Labelling kit (Ambion).

B. Results

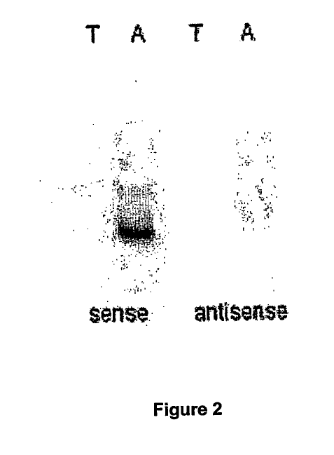

FIG. 1A gives schematic representation of the mechanism that gives rise to the antisense RNA using the conventional oligonucleotides. To determine experimentally if RNA transcribed in the antisense orientation is present in the amplified RNA population using the conventional primers, Northern Blot analysis was performed using oligonucleotide probes complimentary to ubiquitin RNA in sense and antisense orientations (FIG. 2). The oligonucleotide probe complimentary to nucleotides 1691-1953 of ubiquitin mRNA, recognizes 2.3 kb and 1.3 kb bands in the total RNA population which correspond to ubiquitin mRNA isoforms.

Two ubiquitin transcripts are also detected in the amplified RNA population albeit at higher intensity. Since equal mass (10 μg) of total and amplified RNA were used for the Northern Blot analysis, higher signal intensity obtained for the ubiquitin mRNAs in the amplified population indicates the higher level of transcripts per mass in the amplified RNA sample RNA. This is due to the fact that amplification procedure enriches for polyadenylated mRNA species and only 3-5% of total RNA is composed on polyA+RNA. Northern blot analysis performed using a ubiquitin probe complimentary to the antisense transcript reveals presence of antisense ubiquitin transcripts in the amplified RNA population. As expected, ubiquitin transcripts in the antisense orientation are not found in total RNA population. (FIG. 2, right panel, T)

### Example 3

### Utilizing Novel Oligonucleotide Sequence to Block Synthesis of Antisense RNAs

A. Northern Blot Analysis

As in Example 2, RNA was resolved on 1.2% denaturing agarose gel and transferred onto Nylon Membrane using capillary transfer in 10×SSC. After overnight transfer the RNA was cross-linked to the membrane. Overnight hybridization was performed in EXPRESSHYB™ Solution (BD Biosciences Clontech, Palo Alto, Calif., U.S.A.) at 68° C. After hybridization, the membrane washed with low stringency wash 2×SSC, 0.5% SDS at room temperature and high stringency wash 1×SSC, 0.1% SDS at 55° C. and exposed to Phosphoimager screen.

The single-stranded probes used to detect RNAs in the sense and antisense directions were designed to be complementary to ubiquitin NCBI mRNA sequence of human ubiquitin mRNA, GenBank accession #M26880

Ubiquitin Sense Probe

(SEQ ID NO: 5)

5′-GAGAACGTCAAGGCAAAGATCCAGGACAAGGAAGGCATTCCTCCTGA
CCAGCAGAGGTTGATCTTTGCCGGAAAGCAGCTGGAAGATGGGCGGACCC
TGT-3′
Ubiquitin Antisense Probe

(SEQ ID NO: 6)

5′-ACAGGGTCCGCCCATCTTCCAGCTGCTTTCCGGCAAAGATCAACCTC
TGCTGGTCAGGAGGAATGCCTTCCTTGTCCTGGATCTTTGCCTTGACGTT
CTC-3′

Labeling of all double-stranded probes was performed using Deka Prime II kit (Ambion, Woodward, Tex., U.S.A.) using manufacturer's instructions. Labeling of all single-stranded probes were performed using the KINASEMAX™ End Labeling kit (Ambion).

B. Results

As presented in FIG. 1A and demonstrated in Example 2, the redundant oligonucleotide sequence in the conventional primers used to define 3′ and 5′ ends of full-length transcripts allows for the annealing of T7 primer during PCR. Changing the sequence of 64 T containing oligonucleotide in the novel primer CDS64T+ oligo to remove homology to the oligonucleotide used to define the 5′ end of a sequence prevents T7 capswitch primer annealing. This ultimately prevents transcription of ubiquitin in the antisense orientation.

The polyT containing oligonucleotide (CDS 64T) was redesigned to eliminate any homology to the Capswitch primer. The new primer (CDS64T+ oligo) was subsequently used for the RNA amplification.

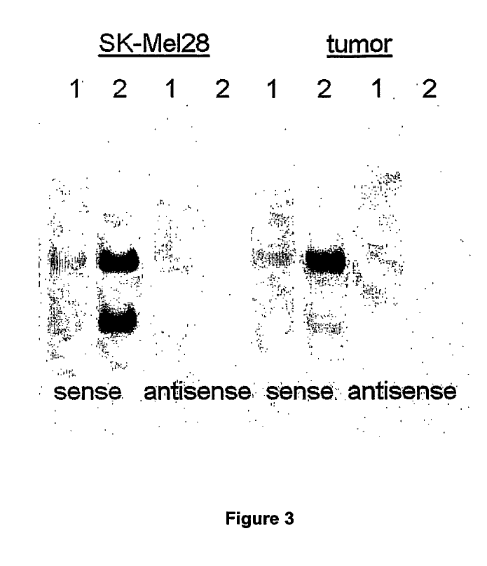

RNAs amplified using CDS 64T and CDS 64T+ oligo were analyzed by Northern Blot analysis. To exclude the possibility that presence of antisense RNA is an artifact of amplification procedure for RNA from a particular cell line, an amplification protocol was also performed on total RNA from a renal cell carcinoma tumor specimen (FIG. 3).

Northern Blot analysis indicates that the redesign of the 64T containing oligonucleotide results in complete block of antisense ubiquitin RNA synthesis in both RNA isolated from a cell line and tumor material. Yields of amplified RNA obtained in amplification procedure using either oligonucleotide was similar, as shown in Table 1.

TABLE 1

| Sample name | A260 | RNA Yield, μg |
|---|---|---|
| SKmel 28 CDS 64T | 0.715 | 75.8 |
| SKmel 28 CDS 64T + oligo | 0.785 | 83.2 |
| RCC CDS 64T | 0.802 | 84.4 |
| RCC CDS 64T + oligo | 0.698 | 74.2 |

### Example 4

### Detection of Double Stranded RNA in Populations Containing Antisense RNA

A. Ribonuclease protection experiments

Ribonuclease protection assays (RPA) were performed using RPA III™ ribonuclease protection assay kit (Ambion, Woodward, Tex., U.S.A.) to determine if double stranded species are present in amplified RNA. In the experiment, 90 ug of 64 T+ oligo or CDS64T amplified RNA was analyzed. Each RNA was analyzed using three experimental conditions: 1) untreated (control), 2) RNAse T1 digest only and 3) RNAse T1 following RNAse III.

For the RPA 30 μg of RNA was ethanol precipitated, reconstituted in 10 ul of RPA III hybridization buffer and incubated overnight with the conventional CDS64T oligonucleotide primer at 56° C. to allow for annealing of complimentary RNA strands. Following incubation, 150 μl of RNase T1 digestion buffer and 8 U of RNase T1 enzyme were added to the samples and incubated at 37° C. for 1 hour. Ethanol precipitation was repeated.

Samples further digested with RNAse III were resuspended in 10 μl of RNAse III digestion buffer and 15 U of the enzyme. After incubation at 37° C. for 1 hr, samples were ethanol precipitated. Control samples contained nuclease-free water instead of either nuclease. Following ethanol precipitations all samples were reconstituted in 20 μl nuclease free water and 1 μl of each sample was analyzed using RNA 6000 Nano chip on an Agilent Bioanalyzer 2100 (Agilent, Palo Alto, Calif., U.S.A.).

B. Results

One concern caused by the presence of antisense RNA is that it may anneal to its coding counterpart resulting in a double stranded RNA. This in turn could lead to deleterious effects such as sequence dependent silencing via an siRNA mediated mechanism. To investigate whether RNA populations amplified using the conventional CDS64T oligonucleotide contain double stranded RNA species, RNAse protection experiments were performed.

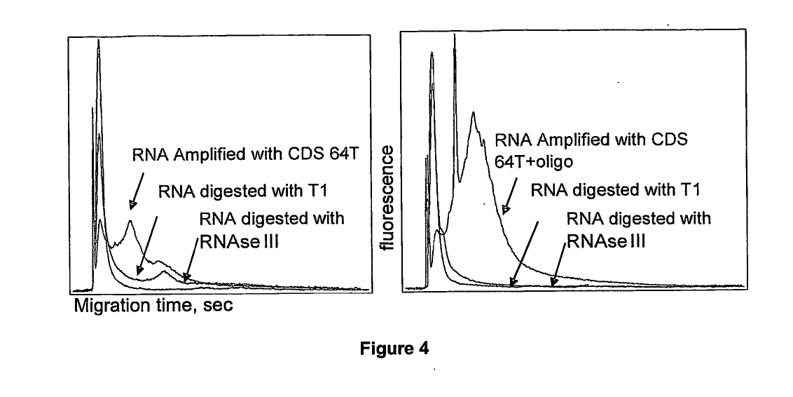

An RNA population amplified using either the conventional (CDS 64T) or novel (CDS 64T+ oligo) poly T containing oligonucleotide was first digested with RNAse T1 specific for single stranded RNA and then further digested with RNAse III specific for double stranded RNA species (FIG. 4).

Amplified RNA migrates as a smear with molecular weight distribution between 200 bp and 6 kb with the peak of intensity around 1.5 kb (FIG. 4, undigested curves). This distribution of molecular weight mass is characteristic of a typical mRNA population. It is also consistent with the contention that only the polyadenylated mRNA population is enriched during the amplification process. After digestion with a single stranded specific RNAse (T1), a single peak of about 400 bp is detected in the population of RNA amplified using CDS 64T (FIG. 4, left panel). Since the material was protected from cleavage by a single stranded RNAse, it is most likely composed of a double stranded RNA species.

To confirm the double stranded RNA nature of the protected population the RNA was further digested with the double stranded specific RNAse III and the peak was completely abolished (FIG. 4, left panel). This further suggests that the 400 bp material is composed of a double stranded RNA.

Notably, no RNA peak after digest with the single stranded specific RNAse T1 is protected in a population amplified using CDS 64T+ oligo (FIG. 4, right panel), suggesting that the amplified population does not contain any double stranded RNA species.

### Example 5

### Examination of Amplified RNA Quality and Fidelity

A. Microarray analysis

Four aliquots 10 μg each of total RNA and four aliquots 2 μg each of amplified RNA from an SK Mel 28 cell line were used in this study. Two aliquots of total RNA and amplified RNA were each labeled using indirect labeling with aminoallyl labeled nucleotides via first strand cDNA synthesis followed by a coupling of the aminoallyl groups to either Cyanine 3 or 5 (Cy 3/Cy5) fluorescent molecules. The remaining total and amplified RNA aliquots were labeled using an indirect labeling protocol with Cy 5 and Cy 3 respectively to perform a dye swap experiment. The labeled cDNAs were hybridized to Agilent Human Genome IA microarrays (Agilent, Palo Alto, Calif., U.S.A.) according to the manufacturing instructions.

The experiment was performed in four replicates where microarray 1 and 2 were hybridized with Cy 3-labeled-total RNA and Cy 5-labeled-amplified RNA. Microarrays 3 and 4 were hybridized with Cy 5-labeled-total RNA and Cy 3-labeled-amplified RNA.

Microarray images were collected using an Axon Scanner. Data analysis was performed using the TIGR TM4 suite, available on the internet at tigr.org. Additional analysis was performed using GeneSpring (Silicon Genetics, Redwood City, Calif., U.S.A.).

B. Results

An initial examination of RNA quality and fidelity amplified using the amplification protocol described in Example 1 was performed using Microarray technology. To evaluate the quality of the experimental data two initial analyses were performed: Cluster and dye-swap.

Hierarchical clustering was used to group the microarrays. In the absence of experimental errors, replicate experiments (i.e., microarrays) will cluster together. The results from the four microarrays showed that microarrays probed with identically labeled RNA populations cluster together: Microarray 1 and 2 cluster together and microarray 3 and 4 cluster together.

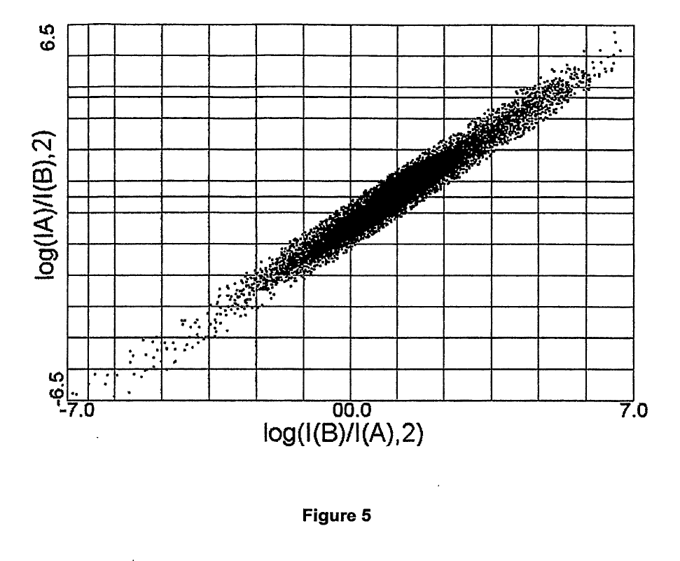

A second test for the quality of microarray data and RNA is to “swap” the cyanine dyes used to label the cDNAs. In the absence of experimental error or labeling bias (i.e., one population of cDNAs preferential labels with one of the dyes), there is a direct correlation when the labeled RNAs are compared. FIG. 5 shows a dye-swap comparison. The genes are tightly grouped and show a direct correlation. The Agilent Human 1A array (Agilent, Palo Alto, Calif., U.S.A.) contains ˜22,575 target genes, 15,742 of which were detected with one RNA population and 15,599 were detected in the other. It is expected that neither population hybridized to all 22,575 genes since the human 1A microarray contains some gene targets that are not expressed in the SK Mel 28 cells.

### Example 6

### Analysis of Yields and Recovery of Cells Transfected with RNA Containing No Antisense Counterpart

A. Generation of DC Cultures and Electroporation with RNA

A leukapheresis from healthy volunteer was collected on a COBE® SPECTRA™ (Gambro BCT, Lakewood, Colo., U.S.A.) using the AutoPBSC procedure by Lifeblood (Memphis, Tenn., U.S.A). Peripheral blood mononuclear cells were isolated from the pheresis using a Ficoll density gradient (HISTOPAQUE®-1007 HYBRI-MAX®, Sigma, St. Louis, Mo., U.S.A.) and cultured for 1-2 hours for adherence of monocytes. The non-adherent cells were removed and the remaining monocytes were cultured in X-VIVO 15™ (Cambrex, East Rutherford, N.J., U.S.A.) medium supplemented with 1000 U/mL of GM-CSF (LEUKINE® liquid, Berlex, Montville, N.J., U.S.A.) and IL-4 (R&D Systems, Minneapolis, Minn., U.S.A.) each for 6-7 days.

The resulting monocyte-derived dendritic cells were harvested, quantified using AO/PI (acridine orange/propidium iodide), and electroporated with 2 or 4 μg RNA per million cells. Transfected cells were cultured overnight in X-VIVO 15 with 800 U/mL GM-CSF, 500 U/mL IL-4, and a maturation cocktail of IL-1β, TNF-α, IL-6, and PGE₂. The mature dendritic cells were harvested and quantified using AO/PI (acridine orange/propidium iodide).

B. Results

To determine if antisense RNA and double stranded RNA cause negative effects, RNA transfection of DCs was performed. Immature DCs were generated from PBMCs and divided into two arms. One experimental arm was transfected with an RNA population from the LNCaP cell line amplified using CDS 64T and a second arm was transfected with RNA amplified using CDS 64T+ oligo. The RNA concentration used in this experiments was 2 μg per million DCs. Cells were placed back into media containing maturation cytokines (TNFα, IL-6 and IL-1β) and PGE2 overnight, harvested and analyzed for phenotypic marker expression and viability.

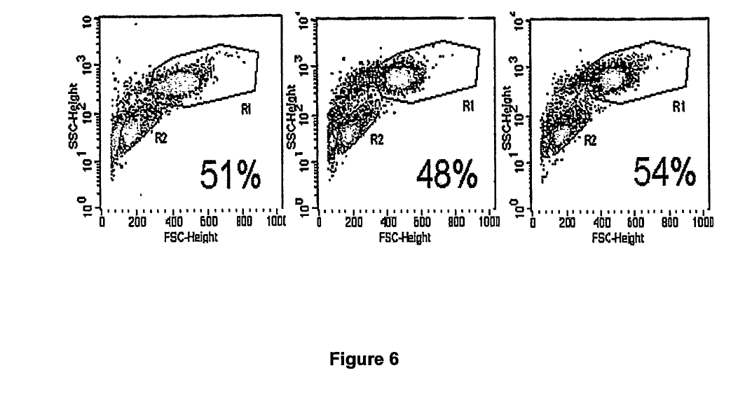

The results indicate that the percentage of viable large cells is higher in cells transfected with RNA amplified using CDS64T+ oligo than in those transfected with RNA amplified using CDS64T (FIG. 6). The population transfected with CDS64T RNA exhibited a pronounced comet-like tail left of the R1 gate. The R1 gate contains a population of large cells, primarily dendritic cells. The comet like tail originating from the R1 gate contains cell debris and dead cells. The tail is less pronounced in a population transfected with RNA amplified using CD64T+ oligo. Further, the number of cells in the R1 gate is larger as well (in this example 54% versus 48%). These observations taken together indicate that the number of viable large cells recovered post-electroporation is higher in the DC population transfected with RNA amplified using the novel oligonucleotide and novel procedure.

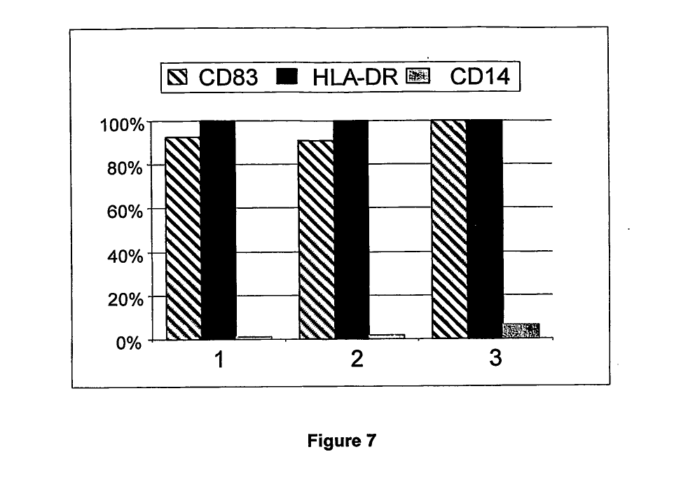

The phenotype of mature viable DCs transfected with RNA amplified using the two procedures was not different (FIG. 7). To compare recovery of viable mature dendritic cells, immature DCs were transfected with different RNAs, matured, harvested and analyzed. Table 2 summarizes data from three large scale experiments performed from leukapharesis material in three independent healthy volunteers. Data summarized in Table 2 demonstrates that total number of cells recovered is greater in the experimental arm transfected with RNA amplified using CDS64T+ oligo than that in the arm transfected with RNA amplified using CDS 64T+ oligo. This indicates that recovery and yield of cells transfected with the novel oligonucleotide and novel method of amplification are higher.

TABLE 2

| | | | CDS 64T + oligo | | |
|---|---|---|---|---|---|
| | CDS 64T | | Cell | | |
| | Cell number | | number | | |
| Experiment | recovered | Est. Doses | recovered | Est. Doses | % Inc. |
| D03-015 | 1.43 × 10⁷ | 6.3 | 2.48 × 10⁷ | 11.1 | 73 |
| D03-018 | 5.04 × 10⁷ | 6.7 | 7.63 × 10⁷ | 10.3 | 51 |
| D03-020 | 3.23 × 10⁷ | 6.0 | 3.9 × 10⁷ | 7.3 | 21 |

### Discussion of Examples

RNA as a vehicle of antigen delivery into DCs overcomes many limitations of DC-based vaccine production. The small amount of tumor from which RNA can be extracted and amplified to generate sufficient quantities for transfection into autologous DCs is one of the advantages. Vaccine trials will allow vaccine preparation and treatment of patients with minimal tumor burden to end-stage disease. Those patients with minimal tumor burden may particularly benefit more from the immunotherapy because they are less immunocompromized.

To generate sufficient quantities of RNA from a small amount of starting material, the amplification protocol discussed herein and elsewhere (Boczkowski et al., 2000 and Heiser et al. 2001 b). The protocol is based on template switching principle and results in amplification of high quality full-length RNA. The Examples demonstrated that the original protocol produces an amplified RNA population that contains antisense RNA.

Antisense RNA is formed because the original protocol was developed for cDNA library construction and required amplification of both sense and antisense cDNA strands. This was achieved by use of a single PCR primer annealing to both 3′ and 5′ ends containing redundant sequences. To allow for the RNA transcription later, the PCR primer contains T7 promoter sequence. As a result when annealed to 3′ end it produces T7 containing cDNA at the 3′ end and sensequensly antisense RNA. Quantitative analysis estimates that 5-7% of the RNA amplified using CDS64T are in the antisense orientation. However, antisense RNA is undetectable in RNA amplified using the novel CDS64T+ oligo. The antisense product is eliminated when redundancy in sequence of 5′ and 3′ oligonucleotides is removed by changing the sequence but not the nucleotide composition MS 64T to CDS64T+ oligo). Alternatively sequence change in a 5′ end-defining (capswitch) oligonucleotide will also prevent antisense RNA formation. Thus, presented herein is experimental evidence for the presence of antisense RNA using the conventional method and oligonucleotide primers. The presently disclosed subject matter devises a solution to eliminate antisense RNA and dsRNA, in part by modifying the primers such that only sense RNA is transcribed.

Microarray analysis was performed to assess the fidelity of RNA amplified using the novel protocol. The amplified RNA was compared to the starting total RNA population using four technical replicates. Cluster analysis grouped microarrays labeled with identically labeled RNAs together: 1 and 2 together and microarray 4 and 3 together. Dye swap experiments revealed tightly grouped genes and a direct correlation between the two groups examined. Both of these analyses confirmed high quality of labeling, hybridization and data extraction. They also confirm the high quality of RNA used in this study.

One of the most significant results from this initial microarray analysis of differential gene expression is that there is only a 0.9% difference (143 genes) between the two RNA populations (total versus amplified: 15,742 and 15,599 respectively). These differences include genes that are detected in total RNA samples and not detected in amplified population (true negatives), and genes not detected in total RNA samples but detected in amplified population (false positives). Both cases may represent bias introduced by the amplification procedure. Most importantly, this number is very small and the vast majority of transcripts in the starting total RNA population are represented in final amplified RNA population. This is indicative of a very high fidelity of the amplified RNA using the developed novel procedure. Therefore, it is demonstrated herein that RNA amplified from total tumor RNA is an accurate representation of the qualitative and quantitative levels of genes expressed in the starting tumor material, thus allowing for a perfectly matched patient-specific vaccine when transfected into autologous DCs.

The Examples further establish a correlation between the presence of antisense RNA and the presence of dsRNA species in the amplified RNA. Using RNAse protection experiments (RPA) it was demonstrated that populations containing antisense RNA also contain double stranded species, and that the loss of antisense RNA is correlated with the loss of double stranded RNAs. The presence of a double stranded RNA in a populations used to transfect DC causes a concern, since mechanisms for dsRNA-mediated silencing are well documented (Heiser et al. 2001 b and Chenchik et al. 1998).

RNAse III used in the RPA experiments has similar sequence specificity as Dicer, an intracellular RNAse responsible for the cleavage of high molecular weight double stranded RNAse into 22-nucleotide double-stranded RNAs. The short 22 nucleotide dsRNA molecules in turn form an active component of an RNA-induced silencing complex (RISC) for sequence-specific gene silencing. Digestion with single strand specific RNAse causes an accumulation of dsRNA species with a molecular weight distribution below 100 nucleotides (FIG. 4, left panel).

Sequence specific RNA-mediated silencing is an evolutionary conserved mechanism found and successfully applied in DC for selective silencing of target genes (Laderach et al., 2003). Thus small dsRNAs formed in DC after transfection with RNA containing large ds RNAs could lead to RISC formation and adversely affect DC phenotype of function.

In experiments aimed at determining whether transfection with RNA containing double stranded species leads to negative effect, DCs from a single donor were generated and then the immature DCs were divided into two arms. Each arm was transfected side by side with the RNA amplified from the same starting total RNA using the original procedure or novel procedure disclosed herein and thus containing ds RNA and no ds RNA, respectively. The study was performed in three distinct donors. The results of this study demonstrated that transfection with RNA that does not contain dsRNA species results in higher cell yield. Since the only difference in the two RNA populations is presence or absence of ds RNA it can be concluded that the decreased yield in the arm transfected with RNA amplified using the original protocol is due to the presence of dsRNA. This is consistent with the prediction discussed herein that ds RNA could lead to deleterious effects on DCs. Viable recovered cells transfected with either RNA population, however, did not exhibit any difference in phenotype.

The data presented herein clearly and surprisingly demonstrate that use of amplified RNA devoid of double stranded species results in higher cell recovery. Therefore transfecting cells with the RNA amplified using the novel protocol and oligonucleotide primers disclosed herein unexpectedly increases the yield of matured, autologous RNA transfected cells, which is directly proportional to the number of doses generated for patient vaccination (Table 2). The increase in cell recovery will therefore reduce the cost of vaccine manufacturing and allow for prolonged patient vaccination, an important advantage over previously known methods.

### REFERENCES

Bender, A. et al. (1996) J. Immunol. Methods, 196, 121.
Berger et al. (2002) J Immunol Methods, 268, 131-40.
Bertoni, R. et al. (1998) J. Immunol., 161, 4447.
Blair, L. et al. 2002 J. Biol. Chem., 277 (1):359-365.
Boczkowski, D., Nair, S. K., Nam, J.-H., Lyerly, H. K, and Gilboa, E. (2000) Cancer Research, 60, 1028-1034.
Boczkowski, D., Nair, S. K., Synder, D. and Gilboa, E. (1996) The Rockefeller University Press, 184, 465-472.
Brenner (1993) Journal of Hematotherapy, 2, 7-17.
Caruso, A. et al. (1997)Cytometry, 27, 71.
Caux, C. et al. (1996) J. Exp. Med., 184, 695.
Chenchik, A., Zhu, Y. Y., Diachenko, L., Li, R., Hill, J. and Siebert, P. D. (1998) In Siebert, P. and Larrick, J. (eds) BioTechniques Books, Natick, Mass., pp. 305-319.
Dykxhoorn, D. M., Novina, C. D., and Sharp, P. A. (2003) Nature Reviews Molecular Cell Biology, 4, 457-467.
European Patent No. 0 320 308.
European Patent No. 0,625,572.
Fay et al. (2000) Blood, 96, 3487.
Fong et al. (2001) J Immunol, 166, 425-44259.
Fujihashi, K. et al. (1993) J. Immunol. Meth., 160, 181.
Grunebach, F., Muller, M. R., Nencioni, A., and Brossart, P. (2003) Gene

Therapy, 10, 367-374.
Haas et al. (1990) Exp. Hematol., 18, 94-98.
Heiser, A., Maurice, M. A., Yancey, D. R., Coleman, D. M., Dahm, P. and Vieweg, J. (2001a) Cancer Research, 61(8), 3388-3393.
Heiser, A., Maurice, M. A., Yancey, D. R., Wu, N. Z., Dham, P., Pruitt, S. K., Boczkowski, D., Nair, S. K., Ballo, M. S., Gilboa, E., and Vieweg, J. (2001 b) The Journal of Immunology, 166, 2953-2960.
Ho, et al. (1995) Stem Cells, 13 (suppl. 3), 100-105.
Hoerr et al. (2000) Eur J Immunol 30, 1-7.
Inaba, et al. (1992) J. Exp. Med., 176, 1693-1702.
Isakov, N. et al. (1995) J. Exp. Med., 181, 375.
Kibler, K. V., Shors, T., Perkins, K. B., Zeman, C. C., Banaszak, M. P., Biesterfeldt, J., Langland, J. O., Jacobs, B. L. (1997) Journal of Virology, 71(3), 1992-2003.
Kobayashi, T., Yamanaka, R., Homma, J., Tsuchiya, N., Yajima, N., Yoshida, S., and Tanaka, R. (2003) Cancer Immunology, Immunotherapy, 52, 632-637 Korbling, M. et al. (1986) Blood, 67, 529-532.
Laderach, D., Compagno, D., Danos, O., Vainchenker, W. and Galy, A. (2003) The Journal of Immunology, 171, 1750-1757.
Lambert et al. (2001) Cancer Res, 61, 641-646.
Lisziewics et al. (2003) Vaccine, 21, 620-623.
Liu et al. (2002) Vaccine 20, 42-48.
MacPherson et al. (1991) PCR: A PRACTICAL APPROACH, IRL Press at Oxford University Press.
Milazzo, C., Reichardt, V. L., Müller, M. R., Grünebach, F. and Brossart, P. (2003) Blood Journal, 101 (3), 977-982
Mitchell, D. A. and Nair, S. K., (2000) Current Opinions in Molecular Therapy, 2(2), 176-181.
Mosier, D. E. et al. (1993) Proc. Natl. Acad. Sci. USA, 90, 2443.
Mu, L. J. Gaudernack, G., Saeboe-Larssen, S., Hammerstad, H., Tierens, A. And Kvalheim, G. (2003) Scand I. Immunol., 58, 578-586.
Mu, L. J. Lazarova, P. Gaudernack, G., Saeboe-Larssen, S. and Kvalheim, G. (2004) Int. J. Immunopath. Pharmacol, 17, 255-264.
Müller, M. R., Grünebach, F., Nencioni, A. and Brossart, P. (2003) The Journal of Immunology, 170, 5892-5896.
Nair, S. and Boczkowski, D. (2002) Expert Rev. Vaccines 1(4), 507-513.
Nencioni, A., Müller, M. R., Grünebach, F., Garuti, A., Mingari, M. C., Patrone, F., Ballestrero, A. and Brossart, P. (2003) Cancer Gene Therapy, 10, 209-214.
O'Hagen (2001) Curr. Drug Targets Infect Disord., 1, 273-286.
Paczesny et al. (2004) J Exp Med., 199, 1503-11.
Parkhurst, M. R. et al. (1996) J. Immunol., 157, 2539.
PCT Published Application No. WO 93/20185.
PCT Published Application No. WO 97/10365.
PCT Published Application No. WO 01/23537.
Ponsaerts, P., Van den Bosch, G., Cools, N., Van Driessche, A., Nijs, G., Lenjou, M., Lardon, F., Van Broeckhoven, C., Van Bockstaele, D. R., Berneman, Z. N. and Van Tenedeloo, V. F. I. (2002) The Journal of Immunology, 169, 1669-1675.
Ponsaerts, P., Van Tendeloo, V. F. I And Berneman, Z. N. (2003) Clin Exp Immunol, 134, 378-384.
Rains, N., Cannan, R. J., Chen, W. and Stubbs, R. S. (2001) Hepato-Gastroenterology, 48, 347-351.
Ribas et al. (2001) Proc Am Soc Clin Onc, 20, 1069.
Romani et al. (1994) J. Exp. Med. 180, 83.
Romani et al. (1996) J. Immunol. Methods, 196, 137.
Salazar, E. et al. (2000) Int. J. Cancer, 85, 829.
Sallusto et al. (1994) J. Exp. Med. 179, 1109-1118.
Schmitt, W. E., Stassar, M. J., Schmitt, W., Little, M. and Cochlovius, B. (2001) Cancer Research Clinical Oncology, 12(3), 203-206.
Schuler-Thurner et al. (2002) J Exp Med., 195, 1279-88. Erratum in: (2003) J Exp Med., 197, 395.
Shirai, M. et al. (1995) J. Immunol., 154, 2733.
Steinman, R. M. (1991) Ann. Rev. Immunol., 9, 271-296.
Stift et al. (2003) J Clin Oncol, 21, 135-142.
Su, Z., Dannull, J., Heiser, A., Yancey, D., Pruitt, S., Madden, J., Coleman, D., Niedzwiecki, D., Gilboa, E. and Vieweg, J. (2003) Cancer Research, 63, 2127-2133.
Szabolcs, et al. (1995) 154, 5851-5861.
Tanquay, S. and Killion, J. J. (1994) Lymphokine Cytokine Res., 13, 259.
Tijssen, P. (ed.) (1993) LABORATORY TECHNIQUES IN BIOCHEMISTRY AND MOLECULAR BIOLOGY, Vol. 24: Hybridization with Nucleic Acid Probes, Elsevier, N.Y.
Utt et al. (1995) Can. J. Microbiol., 41, 152-156.
United States Patent Application No. 20020164346.
United States Patent Application No. 20030032615.
United States Patent Application No. 20030087810.
United States Patent Application No. 20030125242.
United States Patent Application No. 20030199673.
United States Patent Application No. 20040038406.
United States Patent Application No. 20040082530.
U.S. Pat. No. 4,603,102.
U.S. Pat. No. 4,682,195.
U.S. Pat. No. 4,683,202.
U.S. Pat. No. 4,965,188.
U.S. Pat. No. 5,256,536.
U.S. Pat. No. 5,310,652.
U.S. Pat. No. 5,322,770.
U.S. Pat. No. 5,399,491.
U.S. Pat. No. 5,405,783.
U.S. Pat. No. 5,412,087.
U.S. Pat. No. 5,427,202.
U.S. Pat. No. 5,445,934.
U.S. Pat. No. 5,578,832.
U.S. Pat. No. 5,631,734.
U.S. Pat. No. 5,853,719.
U.S. Pat. No. 5,962,271.
U.S. Pat. No. 5,962,272.
U.S. Pat. No. 6,306,388.
U.S. Pat. No. 6,593,086.
U.S. Pat. No. 6,603,998.
U.S. Pat. No. 6,800,734.
U.S. Provisional Patent Application No. 60/522,512.
Velculescu, V. et al. (1995) Science, 270, 484-487.
Ware, C. F. et al. (1983) J. Immunol., 131, 1312.
Yu, et al. (1995) PNAS, 92, 699-703.
Zamore, P. D. and Aronin, N. (2003) Nature Medicine, 9, 347-351.

1. A method of transfecting antigen presenting cells, comprising:
(a) preparing a preparation essentially devoid of antisense-oriented RNA and double-stranded RNA and comprising at least one sense-oriented mRNA encoding an antigen by:
(i) amplifying mRNA from a sample to produce a polynucleotide template, wherein the polynucleotide template comprises a promoter suitable for in vitro transcription operably linked only to a sense strand of the polynucleotide template; and
(ii) in vitro transcribing the polynucleotide template to produce a sense-oriented mRNA encoding an antigen, wherein the polynucleotide template is not a cloned template; and
(b) transfecting antigen presenting cells with the sense-oriented mRNA from the preparation.

2. The method of claim 1, wherein the mRNA in the sample is from a cancer cell or a virion.

3. The method of claim 1, wherein the mRNA in the sample is from a microbial cell.

4. The method of claim 2 wherein the mRNA in the sample is from a cancer cell and the cancer is selected from the group consisting of hematologic malignancies, renal cell cancer, melanoma, breast cancer, prostate cancer, testicular cancer, bladder cancer, ovarian cancer, cervical cancer, stomach cancer, esophageal cancer, pancreatic cancer, lung cancer, neuroblastoma, glioblastoma, retinoblastoma, leukemias, myelomas, lymphomas, hepatoma, adenomas, sarcomas, carcinomas, and blastomas.

5. The method of claim 3, wherein the microbial cell is selected from the group consisting of Helicobacter sp., Salmonella sp., Shigella sp., Enterobacter sp., Campylobacter sp., Mycobacterium sp., Bacillus anthracis, Yersinia pestis, Francisella tularensis, Brucella sp., Leptospira interrogans, Staphylococcus sp., Streptococcus sp., Clostridium sp., Candida albicans, Plasmodium sp., Leishmania sp., and Trypanosoma sp.

6. The method of claim 2, wherein the mRNA in the sample is from a virion selected from the group consisting of human immunodeficiency virus, hepatitis B virus, hepatitis C virus, human papilloma virus, cytomegalovirus, human T-cell lymphotropic virus, herpes simplex virus 1, herpes simplex virus 2, varicella-zoster virus, Epstein-Barr virus, influenza virus, coronavirus, poliomyelitis virus, measles virus, mumps virus, and rubella virus.

7. (canceled)

8. (canceled)

9. (canceled)

10. The method of claim 1, wherein the mRNA in the sample comprises a total mRNA population derived from a cell or a virion.

11. (canceled)

12. (canceled)

13. (canceled)

14. The method of claim 1, wherein amplifying the mRNA from the sample comprises:
(a) reverse transcribing the mRNA from the sample to produce a polynucleotide template comprising a cDNA; and
(b) amplifying the polynucleotide template cDNA using a first primer and a second primer, wherein only one of the first primer and the second primer inserts the promoter suitable for in vitro transcription into the polynucleotide template cDNA.

15. (canceled)

16. (canceled)

17. The method of claim 14, wherein the first and second primers share essentially no sequence homology to one another.

18. The method of claim 14, wherein the first primer comprises a poly T stretch and a 5′ sequence having essentially no sequence homology to the second primer and the second primer comprises the promoter suitable for in vitro transcription.

19. The method of claim 18, wherein the first primer comprises the sequence of SEQ ID NO: 2.

20. The method of claim 1, wherein transfecting is accomplished using a method selected from the group consisting of electroporation, nanoparticle-mediated transfection, peptide-mediated transfection and lipofection.

21. The method of claim 1, wherein the antigen presenting cell is a dendritic cell.

22. The method of claim 21, wherein the dendritic cell is an immature dendritic cell.

23. The method of claim 21 wherein the dendritic cell is a mature dendritic cell.

24. The method of claim 1, wherein the transfecting is in vitro.

25. The method of claim 1, wherein the transfecting is in situ.

26. (canceled)

27. (canceled)

28. (canceled)

29. (canceled)

30. (canceled)

31. (canceled)

32. (canceled)

33. (canceled)

34. A method of generating an immune response in a subject against at least one antigen, comprising introducing a mRNA loaded antigen presenting cell prepared by the method of claim 1 into a subject, wherein the mRNA loaded antigen presenting cell presents the at least one antigen to the immune system of the subject, thereby generating an immune response against the at least one antigen.

35. The method of claim 34, wherein the mRNA encodes at least one antigen from a cancer cell or a virion.

36. The method of claim 35, wherein the mRNA encodes an antigen from a cancer cell or a microbial cell.

37. The method of claim 36 wherein the mRNA encodes an antigen from a cancer cell and the cancer is selected from the group consisting of hematologic malignancies, renal cell cancer, melanoma, breast cancer, prostate cancer, testicular cancer, bladder cancer, ovarian cancer, cervical cancer, stomach cancer, esophageal cancer, pancreatic cancer, lung cancer, neuroblastoma, glioblastoma, retinoblastoma, leukemias, myelomas, lymphomas, hepatoma, adenomas, sarcomas, carcinomas, and blastomas.

38. (canceled)

39. The method of claim 35, wherein the mRNA is from a virion selected from the group consisting of human immunodeficiency virus, hepatitis B virus, hepatitis C virus, human papilloma virus, cytomegalovirus, human T-cell lymphotropic virus, herpes simplex virus 1, herpes simplex virus 2, varicella-zoster virus, Epstein-Barr virus, influenza virus, coronavirus, poliomyelitis virus, measles virus, mumps virus, and rubella virus.

40. (canceled)

41. (canceled)

42. The method of claim 34, wherein the antigen presenting cell is a dendritic cell.

43. (canceled)

44. (canceled)

45. (canceled)
